(19) United States
(12) Patent Application Publication
(10) Pub. No.: US 2022/0351838 A1
(43) Pub. Date: Nov. 3, 2022

(54) METHODS AND SYSTEMS FOR MANAGEMENT AND VISUALIZATION OF RADIOLOGICAL DATA

(71) Applicant: Whiterabbit.ai Inc., Santa Clara, CA (US)

(72) Inventors: Stefano Pedemonte, Santa Clara, CA (US); Arpita Tiwari, Santa Clara, CA (US); Jason Su, Santa Clara, CA (US); Rakesh Mathur, Santa Clara, CA (US); Navarre Joseph, Santa Clara, CA (US)

(21) Appl. No.: 17/711,215

(22) Filed: Apr. 1, 2022

Related U.S. Application Data
(63) Continuation of application No. PCT/US2020/054116, filed on Oct. 2, 2020.
(60) Provisional application No. 62/910,033, filed on Oct. 3, 2019.

(51) Int. Cl. G16H 30/40; G16H 15/00; G06V 10/25; G06T 11/00; G06T 7/00; A61B 6/00

(52) U.S. Cl. CPC G16H 30/40; G16H 15/00; G06V 10/25; G06T 11/00; G06T 7/0012; A61B 6/502; G06V 2201/03; G06T 2207/30068; G06T 2207/30096; G06T 2210/41

(57) ABSTRACT

The present disclosure provides methods and systems directed to management and visualization of radiological data. A method for processing at least one medical image of a location of a body of a subject may comprise (a) retrieving, from a remote server via a network connection, the medical image; (b) identifying one or more regions of interest (ROIs) in the medical image, wherein the ROIs correspond to an anatomical structure of the location of the body of the subject; (c) annotating the ROIs with label information corresponding to the anatomical structure, thereby producing an annotated medical image; (d) generating educational information based at least in part on the annotated medical image; and (e) generating a visualization of the anatomical structure, based at least in part on the educational information.

### CROSS-REFERENCE

The present invention is a continuation of International Patent Application No. PCT/US2020/54116, which claims the benefit of U.S. Provisional Application No. 62/910,033, filed Oct. 3, 2019, which is entirely incorporated herein by reference.

### BACKGROUND

The clinical use of medical imaging examinations, such as routine screening for cancer (e.g., breast cancer), has demonstrated significant benefits in reducing mortality, improving prognoses, and lowering treatment costs. Despite these demonstrated benefits, adoption rates for screening mammography are hindered, in part, by poor patient experience, such as long delays in obtaining an appointment, unclear pricing, long wait times to receive exam results, and confusing reports.

### SUMMARY

The present disclosure provides methods, systems, and media for management and visualization of radiological data, including medical images of subjects. Such subjects may include subjects with a disease, disorder, or abnormal condition (e.g., cancer) and subjects without a disease, disorder, or abnormal condition (e.g., asymptomatic subjects undergoing routine screening exams). The screening may be for a cancer such as, for example, breast cancer.

In an aspect, the present disclosure provides a method for processing at least one medical image of a location of a body of a subject, comprising: (a) retrieving, from a remote server via a network connection, said at least one medical image of said location of said body of said subject; (b) identifying one or more regions of interest (ROIs) in said at least one medical image, wherein said one or more ROIs correspond to at least one anatomical structure of said location of said body of said subject; (c) annotating said one or more ROIs with label information corresponding to said at least one anatomical structure, thereby producing at least one annotated medical image; (d) generating educational information based at least in part on said at least one annotated medical image; and (e) generating a visualization of said at least one anatomical structure of said location of said body of said subject, based at least in part on said educational information.

In some embodiments, said at least one medical image is generated by one or more imaging modalities comprising mammography, a computed tomography (CT) scan, a magnetic resonance imaging (MRI) scan, an ultrasound scan, a chest X-ray, a positron emission tomography (PET) scan, a PET-CT scan, or any combination thereof. In some embodiments, said at least one medical image is generated by mammography. In some embodiments, said location of said body of said subject comprises a breast of said subject. In some embodiments, said one or more ROIs correspond to a lesion of said breast of said subject.

In some embodiments, said remote server comprises a cloud-based server, and wherein said network connection comprises a cloud-based network. In some embodiments, (b) comprises retrieving, from said remote server via said network connection, at least one radiological report corresponding to said at least one medical image, and processing said at least one radiological report to identify said one or more ROIs. In some embodiments, (c) comprises retrieving, from said remote server via said network connection, at least one radiological report corresponding to said at least one medical image, and processing said at least one radiological report to obtain said label information corresponding to said at least one anatomical structure.

In some embodiments, said educational information comprises a location, a definition, a function, a characteristic, or any combination thereof, of said at least one anatomical structure of said location of said body of said subject. In some embodiments, said location comprises a relative location of said at least one anatomical structure with respect to other anatomical structures of said body of said subject. In some embodiments, said other anatomical structures of said body of said subject comprise at least a portion or all of an organ system, an organ, a tissue, a cell, or a combination thereof, of said body of said subject. In some embodiments, said characteristic comprises a density of said at least one anatomical structure. In some embodiments, said educational information comprises diagnostic information, non-diagnostic information, or a combination thereof. In some embodiments, said educational information comprises non-diagnostic information.

In some embodiments, (e) comprises generating said visualization of said at least one anatomical structure on a mobile device of a user. In some embodiments, said method further comprises displaying said visualization of said at least anatomical structure on a display of a user.

In some embodiments, (b) comprises processing said at least one medical image using a trained algorithm to identify said one or more ROIs. In some embodiments, (b) comprises processing said at least one medical image using a trained algorithm to identify said at least one anatomical structure. In some embodiments, (c) comprises processing said one or more ROIs using a trained algorithm to generate said label information. In some embodiments, said trained algorithm comprises a trained machine learning algorithm. In some embodiments, said trained machine learning algorithm comprises a supervised machine learning algorithm. In some embodiments, said supervised machine learning algorithm comprises a deep learning algorithm, a support vector machine (SVM), a neural network, or a Random Forest.

In some embodiments, said at least one medical image is obtained via a routine screening of said subject. In some embodiments, said at least one medical image is obtained as part of a management regimen of a disease, disorder, or abnormal condition of said subject. In some embodiments, said disease, disorder, or abnormal condition is a cancer. In some embodiments, said cancer is breast cancer.

In some embodiments, said method further comprises storing said at least one annotated medical image in a database. In some embodiments, said method further comprises storing said visualization of said at least one anatomical structure in a database.

In another aspect, the present disclosure provides a computer system for processing at least one medical image of a location of a body of a subject, comprising: a database that is configured to store said at least one medical image of said location of said body of said subject; and one or more computer processors operatively coupled to said database, wherein said one or more computer processors are individually or collectively programmed to: (a) retrieve, from a remote server via a network connection, said at least one medical image of said location of said body of said subject; (b) identify one or more regions of interest (ROIs) in said at least one medical image, wherein said one or more ROIs correspond to at least one anatomical structure of said location of said body of said subject; (c) annotate said one or more ROIs with label information corresponding to said at least one anatomical structure, thereby producing at least one annotated medical image; (d) generate educational information based at least in part on said at least one annotated medical image; and (e) generate a visualization of said at least one anatomical structure of said location of said body of said subject, based at least in part on said educational information.

In some embodiments, said at least one medical image is generated by one or more imaging modalities comprising mammography, a computed tomography (CT) scan, a magnetic resonance imaging (MRI) scan, an ultrasound scan, a chest X-ray, a positron emission tomography (PET) scan, a PET-CT scan, or any combination thereof. In some embodiments, said at least one medical image is generated by mammography. In some embodiments, said location of said body of said subject comprises a breast of said subject. In some embodiments, said one or more ROIs correspond to a lesion of said breast of said subject.

In some embodiments, said remote server comprises a cloud-based server, and wherein said network connection comprises a cloud-based network. In some embodiments, (b) comprises retrieving, from said remote server via said network connection, at least one radiological report corresponding to said at least one medical image, and processing said at least one radiological report to identify said one or more ROIs. In some embodiments, (c) comprises retrieving, from said remote server via said network connection, at least one radiological report corresponding to said at least one medical image, and processing said at least one radiological report to obtain said label information corresponding to said at least one anatomical structure.

In some embodiments, said educational information comprises a location, a definition, a function, a characteristic, or any combination thereof, of said at least one anatomical structure of said location of said body of said subject. In some embodiments, said location comprises a relative location of said at least one anatomical structure with respect to other anatomical structures of said body of said subject. In some embodiments, said other anatomical structures of said body of said subject comprise at least a portion or all of an organ system, an organ, a tissue, a cell, or a combination thereof, of said body of said subject. In some embodiments, said characteristic comprises a density of said at least one anatomical structure. In some embodiments, said educational information comprises diagnostic information, non-diagnostic information, or a combination thereof. In some embodiments, said educational information comprises non-diagnostic information.

In some embodiments, (e) comprises generating said visualization of said at least one anatomical structure on a mobile device of a user. In some embodiments, said one or more computer processors are individually or collectively programmed to further display said visualization of said at least anatomical structure on a display of a user.

In some embodiments, (b) comprises processing said at least one medical image using a trained algorithm to identify said one or more ROIs. In some embodiments, (b) comprises processing said at least one medical image using a trained algorithm to identify said at least one anatomical structure. In some embodiments, (c) comprises processing said one or more ROIs using a trained algorithm to generate said label information. In some embodiments, said trained algorithm comprises a trained machine learning algorithm. In some embodiments, said trained machine learning algorithm comprises a supervised machine learning algorithm. In some embodiments, said supervised machine learning algorithm comprises a deep learning algorithm, a support vector machine (SVM), a neural network, or a Random Forest.

In some embodiments, said at least one medical image is obtained via a routine screening of said subject. In some embodiments, said at least one medical image is obtained as part of a management regimen of a disease, disorder, or abnormal condition of said subject. In some embodiments, said disease, disorder, or abnormal condition is a cancer. In some embodiments, said cancer is breast cancer.

In some embodiments, said one or more computer processors are individually or collectively programmed to further store said at least one annotated medical image in a database. In some embodiments, said one or more computer processors are individually or collectively programmed to further store said visualization of said at least one anatomical structure in a database.

In another aspect, the present disclosure provides a non-transitory computer readable medium comprising machine-executable code that, upon execution by one or more computer processors, implements a method for processing at least one medical image of a location of a body of a subject, said method comprising: (a) retrieving, from a remote server via a network connection, said at least one medical image of said location of said body of said subject; (b) identifying one or more regions of interest (ROIs) in said at least one medical image, wherein said one or more ROIs correspond to at least one anatomical structure of said location of said body of said subject; (c) annotating said one or more ROIs with label information corresponding to said at least one anatomical structure, thereby producing at least one annotated medical image; (d) generating educational information based at least in part on said at least one annotated medical image; and (e) generating a visualization of said at least one anatomical structure of said location of said body of said subject, based at least in part on said educational information.

In some embodiments, said at least one medical image is generated by one or more imaging modalities comprising mammography, a computed tomography (CT) scan, a magnetic resonance imaging (MRI) scan, an ultrasound scan, a chest X-ray, a positron emission tomography (PET) scan, a PET-CT scan, or any combination thereof. In some embodiments, said at least one medical image is generated by mammography. In some embodiments, said location of said body of said subject comprises a breast of said subject. In some embodiments, said one or more ROIs correspond to a lesion of said breast of said subject.

In some embodiments, said remote server comprises a cloud-based server, and wherein said network connection comprises a cloud-based network. In some embodiments, (b) comprises retrieving, from said remote server via said network connection, at least one radiological report corresponding to said at least one medical image, and processing said at least one radiological report to identify said one or more ROIs. In some embodiments, (c) comprises retrieving, from said remote server via said network connection, at least one radiological report corresponding to said at least one medical image, and processing said at least one radiological report to obtain said label information corresponding to said at least one anatomical structure.

In some embodiments, said educational information comprises a location, a definition, a function, a characteristic, or any combination thereof, of said at least one anatomical structure of said location of said body of said subject. In some embodiments, said location comprises a relative location of said at least one anatomical structure with respect to other anatomical structures of said body of said subject. In some embodiments, said other anatomical structures of said body of said subject comprise at least a portion or all of an organ system, an organ, a tissue, a cell, or a combination thereof, of said body of said subject. In some embodiments, said characteristic comprises a density of said at least one anatomical structure. In some embodiments, said educational information comprises diagnostic information, non-diagnostic information, or a combination thereof. In some embodiments, said educational information comprises non-diagnostic information.

In some embodiments, (e) comprises generating said visualization of said at least one anatomical structure on a mobile device of a user. In some embodiments, said method of said non-transitory computer readable medium further comprises displaying said visualization of said at least anatomical structure on a display of a user.

In some embodiments, (b) comprises processing said at least one medical image using a trained algorithm to identify said one or more ROIs. In some embodiments, (b) comprises processing said at least one medical image using a trained algorithm to identify said at least one anatomical structure. In some embodiments, (c) comprises processing said one or more ROIs using a trained algorithm to generate said label information. In some embodiments, said trained algorithm comprises a trained machine learning algorithm. In some embodiments, said trained machine learning algorithm comprises a supervised machine learning algorithm. In some embodiments, said supervised machine learning algorithm comprises a deep learning algorithm, a support vector machine (SVM), a neural network, or a Random Forest.

In some embodiments, said at least one medical image is obtained via a routine screening of said subject. In some embodiments, said at least one medical image is obtained as part of a management regimen of a disease, disorder, or abnormal condition of said subject. In some embodiments, said disease, disorder, or abnormal condition is a cancer. In some embodiments, said cancer is breast cancer.

In some embodiments, said method of said non-transitory computer readable medium further comprises storing said at least one annotated medical image in a database. In some embodiments, said method of said non-transitory computer readable medium further comprises storing said visualization of said at least one anatomical structure in a database.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example workflow of a method for radiological data management and visualization, in accordance with disclosed embodiments.

FIG. 2 illustrates a computer system that is programmed or otherwise configured to implement methods provided herein.

FIG. 3A shows an example screenshot of a mobile application of a radiological data management and visualization system, in accordance with disclosed embodiments. The mobile application is configured to allow a user to participate in the account creation process, which may comprise signing up as a user of the mobile application, or to sign in to the mobile application as an existing registered user of the mobile application.

FIG. 3B shows an example screenshot of a mobile application of a radiological data management and visualization system, in accordance with disclosed embodiments. The mobile application is configured to allow a patient to create a user account of the radiological data management and visualization system, by entering an e-mail address or phone number and creating a password.

FIG. 3C shows an example screenshot of a mobile application of a radiological data management and visualization system, in accordance with disclosed embodiments. The mobile application is configured to allow a user to participate in the patient verification process, which may comprise providing personal information (e.g., first name, last name, date of birth, and last 4 digits of phone number) to identify himself or herself as a patient of an in-network clinic of the radiological data management and visualization system.

FIGS. 3D-3E show example screenshots of a mobile application of a radiological data management and visualization system, in accordance with disclosed embodiments. The mobile application is configured to authenticate a user by sending a verification code to the user (e.g., through a text message to a phone number of the user) and receiving user input of the verification code.

FIG. 4A-4B show example screenshots of a mobile application of a radiological data management and visualization system, in accordance with disclosed embodiments. The mobile application is configured to allow a user (e.g., a patient) to view a list of his or her appointments.

FIGS. 4C-4D show example screenshots of a mobile application of a radiological data management and visualization system, in accordance with disclosed embodiments. The mobile application is configured to allow a user (e.g., a patient) to book an appointment for radiological assessment (e.g., radiological screening such as mammography).

FIG. 4E shows an example screenshot of a mobile application of a radiological data management and visualization system, in accordance with disclosed embodiments. The mobile application is configured to allow a patient to participate in a pre-screening check, in which the user is provided a series of questions and is prompted to input response to the series of questions.

FIG. 4F shows an example of a screenshot of a mobile application of a radiological data management and visualization system, in accordance with disclosed embodiments. The mobile application is configured to allow a user (e.g., a patient) to view a list of his or her appointments.

FIGS. 4G-4H show example screenshots of a mobile application of a radiological data management and visualization system, in accordance with disclosed embodiments. The mobile application is configured to allow a user (e.g., a patient) to enter his or her personal information (e.g., name, address, sex, and date of birth) into a fillable form.

FIG. 4I shows an example of a screenshot of a mobile application of a radiological data management and visualization system, in accordance with disclosed embodiments. The mobile application is configured to present a user (e.g., a patient) with a fillable form (e.g., a questionnaire such as a breast imaging questionnaire) and to allow the user to input information in response to the questionnaire.

FIG. 4J shows an example of a screenshot of a mobile application of a radiological data management and visualization system, in accordance with disclosed embodiments. The mobile application is configured to present a user (e.g., a patient) with a confirmation that his or her information has been updated, and to link the user to the “My Images” page to view his or her complete record of radiology images.

FIG. 5A shows an example of a screenshot of a mobile application of a radiological data management and visualization system, in accordance with disclosed embodiments. The mobile application provides an image viewer configured to allow a user (e.g., a patient) to view sets of his or her medical images (e.g., through a “My Images” page of the mobile application) that have been acquired and stored.

FIG. 5B shows an example of a screenshot of a mobile application of a radiological data management and visualization system, in accordance with disclosed embodiments. The mobile application provides an image viewer configured to allow a user (e.g., a patient) to view details of a given medical image upon selection.

FIG. 5C shows an example of a screenshot of a mobile application of a radiological data management and visualization system, in accordance with disclosed embodiments. The mobile application provides an image viewer configured to allow a user (e.g., a patient) to view details of a given medical image upon selection.

FIG. 5D shows an example of a screenshot of a mobile application of a radiological data management and visualization system, in accordance with disclosed embodiments. The mobile application provides an image viewer configured to allow a user (e.g., a patient) to view details of a given medical image upon selection.

FIG. 5E shows an example of a screenshot of a mobile application of a radiological data management and visualization system, in accordance with disclosed embodiments. The mobile application is configured to allow a user (e.g., a patient) to view details of a given medical image that has been acquired and stored, such as annotation options.

FIGS. 6A-6B show example screenshots of a mobile application of a radiological data management and visualization system, in accordance with disclosed embodiments. The mobile application is configured to allow a user (e.g., a patient) to share his or her exams (e.g., including medical image data and/or reports) to other parties (e.g., physicians or other clinical health providers, family members, or friends), such as by clicking a “Share” button from the “My Images” page.

FIGS. 7A-7S show example screenshots of a mobile application of a radiological data management and visualization system, in accordance with disclosed embodiments. The mobile application is configured to allow a user (e.g., a patient) to book a dual radiological exam (e.g., mammogram and MRI) and facilitate the patient experience throughout the exam process.

FIGS. 8A-8H show examples of screenshots of a mobile application showing mammogram reports.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject can be a person that has a disease, disorder, or abnormal condition (e.g., cancer) or is suspected of having a disease, disorder, or abnormal condition. The subject may be displaying a symptom(s) indicative of a health or physiological state or condition of the subject, such as a cancer (e.g., breast cancer) of the subject. As an alternative, the subject can be asymptomatic with respect to such health or physiological state or condition.

The clinical use of medical imaging examinations, such as routine screening for cancer (e.g., breast cancer), has demonstrated significant benefits in reducing mortality, improving prognoses, and lowering treatment costs. Despite these demonstrated benefits, adoption rates for screening mammography are hindered, in part, by poor patient experience, such as long delays in obtaining an appointment, unclear pricing, long wait times to receive exam results, and confusing reports.

The present disclosure provides methods, systems, and media for management and visualization of radiological data, including medical images of subjects. Such subjects may include subjects with a disease, disorder, or abnormal condition (e.g., cancer) and subjects without a disease, disorder, or abnormal condition (e.g., asymptomatic subjects undergoing routine screening exams). The screening may be for a cancer such as, for example, breast cancer.

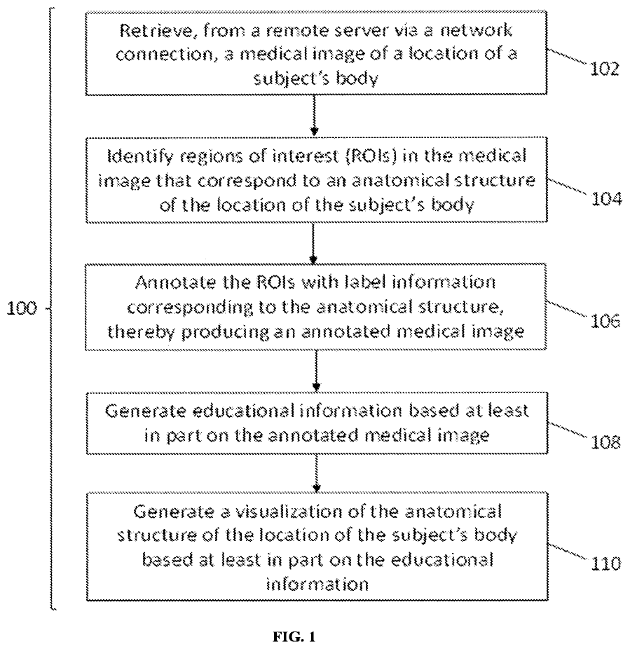

FIG. 1 illustrates an example workflow of a method for radiological data management and visualization, in accordance with disclosed embodiments. In an aspect, the present disclosure provides a method 100 for processing at least one image of a location of a body of a subject. The method 100 may comprise retrieving, from a remote server via a network connection, a medical image of a location of a subject's body (as in operation 102). Next, the method 100 may comprise identifying regions of interest (ROIs) in the medical image that correspond to an anatomical structure of the location of the subject's body (as in operation 104). For example, the ROIs may be identified by applying a trained algorithm to the medical image. Next, the method 100 may comprise annotating the ROIs with label information corresponding to the anatomical structure, thereby producing an annotated medical image (as in operation 106). Next, the method 100 may comprise generating educational information based at least in part on the annotated medical image (as in operation 108). Next, the method 100 may comprise generating a visualization of the anatomical structure of the location of the subject's body based at least in part on the educational information (as in operation 110).

### Obtaining Medical Images

A set of one or more medical images may be obtained or derived from a human subject (e.g., a patient). The medical images may be stored in a database, such as a computer server (e.g., cloud-based server), a local server, a local computer, or a mobile device (such as smartphone or tablet)). The medical images may be obtained from a subject with a disease, disorder, or abnormal condition, from a subject that is suspected of having the disease, disorder, or abnormal condition, or from a subject that does not have or is not suspected of having the disease, disorder, or abnormal condition.

The medical images may be taken before and/or after treatment of a subject with a disease, disorder, or abnormal condition. Medical images may be obtained from a subject during a treatment or a treatment regime. Multiple sets of medical images may be obtained from a subject to monitor the effects of the treatment over time. The medical images may be taken from a subject known or suspected of having a disease, disorder, or abnormal condition (e.g., cancer such as breast cancer) for which a definitive positive or negative diagnosis is not available via clinical tests. The medical images may be taken from a subject suspected of having a disease, disorder, or abnormal condition. The medical images may be taken from a subject experiencing unexplained symptoms, such as fatigue, nausea, weight loss, aches and pains, weakness, or bleeding. The medical images may be taken from a subject having explained symptoms. The medical images may be taken from a subject at risk of developing a disease, disorder, or abnormal condition due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The medical images may be acquired using one or more imaging modalities, such as a mammography, a computed tomography (CT) scan, a magnetic resonance imaging (MRI) scan, an ultrasound scan, a chest X-ray, a positron emission tomography (PET) scan, a PET-CT scan, or any combination thereof. The medical images may be pre-processed using image processing techniques to enhance image characteristics (e.g., contrast, brightness, sharpness), remove noise or artifacts, filter frequency ranges, compress the images to a small file size, or sample or crop the images. The medical images may be deconstructed or reconstructed (e.g., to create a 3-D rendering from a plurality of 2-D images).

### Trained Algorithms

After obtaining medical images of a location of a body of a subject, one or more trained algorithms may be used to process the medical images to (i) identify regions of interest (ROIs) in the medical images that correspond to anatomical structures of the location of the body of the subject, (ii) identify the anatomical structures of the location of the body of the subject, (iii) generate label information of the anatomical structures, or (iv) a combination thereof. The trained algorithm may be configured to generate the outputs (e.g., the ROIs or anatomical structures) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network (e.g., a deep neural network (DNN)), or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise features extracted from one or more datasets comprising medical images of a location of a body of a subject. For example, an input variable may comprise a number of potentially diseased or cancerous or suspicious regions of interest (ROIs) in the dataset of medical images. The potentially diseased or cancerous or suspicious regions of interest (ROIs) may be identified or extracted from the dataset of medical images using a variety of image processing approaches, such as image segmentation. The plurality of input variables may also include clinical health data of a subject.

In some embodiments, the clinical health data comprises one or more quantitative measures of the subject, such as age, weight, height, body mass index (BMI), blood pressure, heart rate, glucose levels. As another example, the clinical health data can comprise one or more categorical measures, such as race, ethnicity, history of medication or other clinical treatment, history of tobacco use, history of alcohol consumption, daily activity or fitness level, genetic test results, blood test results, imaging results, and screening results.

The trained algorithm may comprise a classifier, such that each of the one or more output values comprises one of a fixed number of possible values (e.g., a linear classifier, a logistic regression classifier, etc.) indicating a classification of the datasets comprising medical images by the classifier. The trained algorithm may comprise a binary classifier, such that each of the one or more output values comprises one of two values (e.g., {0, 1}, {positive, negative}, {high-risk, low-risk}, or {suspicious, normal}) indicating a classification of the datasets comprising medical images by the classifier. The trained algorithm may be another type of classifier, such that each of the one or more output values comprises one of more than two values (e.g., {0, 1, 2}, {positive, negative, or indeterminate}, {high-risk, intermediate-risk, or low-risk}, or {suspicious, normal, or indeterminate}) indicating a classification of the datasets comprising medical images by the classifier. The output values may comprise descriptive labels, numerical values, or a combination thereof. Some of the output values may comprise descriptive labels. Such descriptive labels may provide an identification, indication, likelihood, or risk of a disease or disorder state of the subject, and may comprise, for example, positive, negative, high-risk, intermediate-risk, low-risk, suspicious, normal, or indeterminate. Such descriptive labels may provide label information for annotation, which corresponds to anatomical structures of the location of the body of the subject. Such descriptive labels may provide an identification of a follow-up diagnostic procedure or treatment for the subject, and may comprise, for example, a therapeutic intervention, a duration of the therapeutic intervention, and/or a dosage of the therapeutic intervention suitable to treat a disease, disorder, or abnormal condition or other condition. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. As another example, such descriptive labels may provide a prognosis of the disease, disorder, or abnormal condition of the subject. As another example, such descriptive labels may provide a relative assessment of the disease, disorder, or abnormal condition (e.g., an estimated cancer stage or tumor burden) of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the disease, disorder, or abnormal condition of the subject. Some numerical values may be mapped to descriptive labels, for example, by mapping 1 to “positive” and 0 to “negative.”

Some of the output values may be assigned based on one or more cutoff values. For example, a binary classification of medical images may assign an output value of “positive” or 1 if the analysis of the medical image indicates that the medical image has at least a 50% probability of having a suspicious ROI. For example, a binary classification of medical images may assign an output value of “negative” or 0 if the analysis of the medical image indicates that the medical image has less than a 50% probability of having a suspicious ROI. In this case, a single cutoff value of 50% is used to classify medical images into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of medical images may assign an output value of “positive” or 1 if the analysis of the medical image indicates that the medical image has a probability of having a suspicious ROI of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of medical images may assign an output value of “positive” or 1 if the analysis of the medical image indicates that the medical image has a probability of having a suspicious ROI of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of medical images may assign an output value of “negative” or 0 if the analysis of the medical image indicates that the medical image has a probability of having a suspicious ROI of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%. The classification of medical images may assign an output value of “negative” or 0 if the analysis of the medical image indicates that the medical image has a probability of having a suspicious ROI of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%.

The classification of medical images may assign an output value of “indeterminate” or 2 if the medical image is not classified as “positive”, “negative”, 1, or 0. In this case, a set of two cutoff values is used to classify medical images into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify medical images into one of n+1 possible output values, where n is any positive integer.

The trained algorithm may be trained with a plurality of independent training samples. Each of the independent training samples may comprise a set of medical images from a subject, associated datasets obtained by analyzing the medical images (e.g., labels or annotations), and one or more known output values corresponding to the sets of medical images (e.g., a set of suspicious ROIs, a clinical diagnosis, prognosis, absence, or treatment or efficacy of a disease, disorder, or abnormal condition of the subject). Independent training samples may comprise medical images, and associated datasets and outputs obtained or derived from a plurality of different subjects. Independent training samples may comprise medical images and associated datasets and outputs obtained at a plurality of different time points from the same subject (e.g., on a regular basis such as weekly, biweekly, or monthly). Independent training samples may be associated with presence of the suspicious ROIs or the disease, disorder, or abnormal condition (e.g., training samples comprising dataset comprising medical images, and associated datasets and outputs obtained or derived from a plurality of subjects known to have the suspicious ROIs or the disease, disorder, or abnormal condition). Independent training samples may be associated with absence of the suspicious ROIs or the disease, disorder, or abnormal condition (e.g., training samples comprising dataset comprising medical images, and associated datasets and outputs obtained or derived from a plurality of subjects who are known to not have a previous diagnosis of the disease, disorder, or abnormal condition or who have received a negative test result for the suspicious ROIs or the disease, disorder, or abnormal condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise medical images associated with presence of the suspicious ROIs or the disease, disorder, or abnormal condition and/or medical images associated with absence of the suspicious ROIs or the disease, disorder, or abnormal condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the suspicious ROIs or the disease, disorder, or abnormal condition. In some embodiments, the dataset comprising medical images is independent of samples used to train the trained algorithm.

The trained algorithm may be trained with a first number of independent training samples associated with presence of the suspicious ROIs or the disease, disorder, or abnormal condition and a second number of independent training samples associated with absence of the suspicious ROIs or the disease, disorder, or abnormal condition. The first number of independent training samples associated with presence of the suspicious ROIs or the disease, disorder, or abnormal condition may be no more than the second number of independent training samples associated with absence of the suspicious ROIs or the disease, disorder, or abnormal condition. The first number of independent training samples associated with presence of the suspicious ROIs or the disease, disorder, or abnormal condition may be equal to the second number of independent training samples associated with absence of the suspicious ROIs or the disease, disorder, or abnormal condition. The first number of independent training samples associated with presence of the suspicious ROIs or the disease, disorder, or abnormal condition may be greater than the second number of independent training samples associated with absence of the suspicious ROIs or the disease, disorder, or abnormal condition.

The trained algorithm may be configured to generate the outputs (e.g., the ROIs or anatomical structures) with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of generating the outputs (e.g., the ROIs or anatomical structures) by the trained algorithm may be calculated as the percentage of independent test samples (e.g., images from subjects known to have the suspicious ROIs or subjects with negative clinical test results for the suspicious ROIs) that are correctly identified or classified as being normal or suspicious.

The trained algorithm may be configured to generate the outputs (e.g., the ROIs or anatomical structures) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of generating the outputs (e.g., the ROIs or anatomical structures) using the trained algorithm may be calculated as the percentage of medical images identified or classified as having suspicious ROIs that correspond to subjects that truly have a suspicious ROI.

The trained algorithm may be configured to generate the outputs (e.g., the ROIs or anatomical structures) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of generating the outputs (e.g., the ROIs or anatomical structures) using the trained algorithm may be calculated as the percentage of medical images identified or classified as being normal that correspond to subjects that truly do not have a suspicious ROI.

The trained algorithm may be configured to generate the outputs (e.g., the ROIs or anatomical structures) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of generate the outputs (e.g., the ROIs or anatomical structures) using the trained algorithm may be calculated as the percentage of medical images obtained from subjects known to have a suspicious ROI that are correctly identified or classified as having suspicious ROIs.

The trained algorithm may be configured to generate the outputs (e.g., the ROIs or anatomical structures) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of generate the outputs (e.g., the ROIs or anatomical structures) using the trained algorithm may be calculated as the percentage of medical images obtained from subjects without a suspicious ROI (e.g., subjects with negative clinical test results) that are correctly identified or classified as not having suspicious ROIs.

The trained algorithm may be configured to generate the outputs (e.g., the ROIs or anatomical structures) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operating Characteristic (ROC) curve (e.g., the area under the ROC curve) associated with the trained algorithm in generating the outputs (e.g., the ROIs or anatomical structures).

The trained algorithm may be adjusted or tuned to improve one or more of the performance, accuracy, PPV, NPV, clinical sensitivity, clinical specificity, or AUC of generating the outputs (e.g., the ROIs or anatomical structures). The trained algorithm may be adjusted or tuned by adjusting parameters of the trained algorithm (e.g., a set of cutoff values used to classify medical images as described elsewhere herein, or parameters or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of features of the medical images may be identified as most influential or most important to be included for making high-quality classifications or identifications of ROIs or anatomical structures. The plurality of features of the medical images or a subset thereof may be ranked based on classification metrics indicative of each individual feature's influence or importance toward making high-quality classifications or identifications of ROIs or anatomical structures. Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Identifying or Monitoring Suspicious ROIs

After using a trained algorithm to process the medical images of a location of a body of a subject to generate the outputs (e.g., identifications of ROIs or anatomical structures), the subject may be monitored over a duration of time. The monitoring may be performed based at least in part on the generated outputs (e.g., identifications of ROIs or anatomical structures), a plurality of features extracted from the medical images, and/or clinical health data of the subject. The monitoring decisions may be made by a radiologist, a plurality of radiologists, or a trained algorithm.

In some embodiments, the subject may be identified as being at risk of a disease, disorder, or abnormal condition (e.g., cancer) based on the identifications of ROIs or anatomical structures. After identifying the subject as being at risk of a disease, disorder, or abnormal condition, a clinical intervention for the subject may be selected based at least in part on the disease, disorder, or abnormal condition for which the subject is identified as being at risk. In some embodiments, the clinical intervention is selected from a plurality of clinical interventions (e.g., clinically indicated for different types of the disease, disorder, or abnormal condition).

In some embodiments, the trained algorithm may determine that the subject is at risk of a disease, disorder, or abnormal condition of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of a disease, disorder, or abnormal condition at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the disease, disorder, or abnormal condition (e.g., cancer), the subject may be optionally provided with a therapeutic intervention (e.g., prescribing an appropriate course of treatment to treat the disease, disorder, or abnormal condition of the subject). The therapeutic intervention may comprise a prescription of an effective dose of a drug, a further testing or evaluation of the disease, disorder, or abnormal condition, a further monitoring of the disease, disorder, or abnormal condition, or a combination thereof. If the subject is currently being treated for the disease, disorder, or abnormal condition with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the disease, disorder, or abnormal condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The identifications of ROIs or anatomical structures, a plurality of features extracted from the medical images; and/or clinical health data of the subject may be assessed over a duration of time to monitor a patient (e.g., subject who has a disease, disorder, or abnormal condition, who is suspected of having a disease, disorder, or abnormal condition, or who is being treated for a disease, disorder, or abnormal condition). In some cases, the identifications of ROIs or anatomical structures in the medical images of the patient may change during the course of treatment. For example, the features of the medical images of a patient with decreasing risk of the disease, disorder, or abnormal condition due to an effective treatment may shift toward the profile or distribution of a healthy subject (e.g., a subject without the disease, disorder, or abnormal condition). Conversely, for example, the features of the medical images of a patient with increasing risk of the disease, disorder, or abnormal condition due to an ineffective treatment may shift toward the profile or distribution of a subject with higher risk of the disease, disorder, or abnormal condition or a more advanced form of the disease, disorder, or abnormal condition.

The subject may be monitored by monitoring a course of treatment for treating the disease, disorder, or abnormal condition of the subject. The monitoring may comprise assessing the disease, disorder, or abnormal condition of the subject at two or more time points. The assessing may be based at least on the identifications of ROIs or anatomical structures, a plurality of features extracted from the medical images; and/or clinical health data of the subject determined at each of the two or more time points.

In some embodiments, a difference in the identifications of ROIs or anatomical structures, a plurality of features extracted from the medical images; and/or clinical health data of the subject between the two or more time points may be indicative of one or more clinical indications, such as (i) a diagnosis of the disease, disorder, or abnormal condition of the subject, (ii) a prognosis of the disease, disorder, or abnormal condition of the subject, (iii) an increased risk of the disease, disorder, or abnormal condition of the subject, (iv) a decreased risk of the disease, disorder, or abnormal condition of the subject, (v) an efficacy of the course of treatment for treating the disease, disorder, or abnormal condition of the subject, and (vi) a non-efficacy of the course of treatment for treating the disease, disorder, or abnormal condition of the subject.

In some embodiments, a difference in the identifications of ROIs or anatomical structures, a plurality of features extracted from the medical images; and/or clinical health data of the subject between the two or more time points may be indicative of a diagnosis of the disease, disorder, or abnormal condition of the subject. For example, if the disease, disorder, or abnormal condition was not detected in the subject at an earlier time point but was detected in the subject at a later time point, then the difference is indicative of a diagnosis of the disease, disorder, or abnormal condition of the subject. A clinical action or decision may be made based on this indication of diagnosis of the disease, disorder, or abnormal condition of the subject, such as, for example, prescribing a new therapeutic intervention for the subject. The clinical action or decision may comprise recommending the subject for a secondary clinical test to confirm the diagnosis of the disease, disorder, or abnormal condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the identifications of ROIs or anatomical structures, a plurality of features extracted from the medical images; and/or clinical health data of the subject between the two or more time points may be indicative of a prognosis of the disease, disorder, or abnormal condition of the subject.

In some embodiments, a difference in the identifications of ROIs or anatomical structures, a plurality of features extracted from the medical images; and/or clinical health data of the subject between the two or more time points may be indicative of the subject having an increased risk of the disease, disorder, or abnormal condition. For example, if the disease, disorder, or abnormal condition was detected in the subject both at an earlier time point and at a later time point, and if the difference is a positive difference (e.g., an increase from the earlier time point to the later time point), then the difference may be indicative of the subject having an increased risk of the disease, disorder, or abnormal condition. A clinical action or decision may be made based on this indication of the increased risk of the disease, disorder, or abnormal condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the disease, disorder, or abnormal condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the identifications of ROIs or anatomical structures, a plurality of features extracted from the medical images; and/or clinical health data of the subject between the two or more time points may be indicative of the subject having a decreased risk of the disease, disorder, or abnormal condition. For example, if the disease, disorder, or abnormal condition was detected in the subject both at an earlier time point and at a later time point, and if the difference is a negative difference (e.g., a decrease from the earlier time point to the later time point), then the difference may be indicative of the subject having a decreased risk of the disease, disorder, or abnormal condition. A clinical action or decision may be made based on this indication of the decreased risk of the disease, disorder, or abnormal condition (e.g., continuing or ending a current therapeutic intervention) for the subject. The clinical action or decision may comprise recommending the subject for a secondary clinical test to confirm the decreased risk of the disease, disorder, or abnormal condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the identifications of ROIs or anatomical structures, a plurality of features extracted from the medical images; and/or clinical health data of the subject between the two or more time points may be indicative of an efficacy of the course of treatment for treating the disease, disorder, or abnormal condition of the subject. For example, if the disease, disorder, or abnormal condition was detected in the subject at an earlier time point but was not detected in the subject at a later time point, then the difference may be indicative of an efficacy of the course of treatment for treating the disease, disorder, or abnormal condition of the subject. A clinical action or decision may be made based on this indication of the efficacy of the course of treatment for treating the disease, disorder, or abnormal condition of the subject, e.g., continuing or ending a current therapeutic intervention for the subject. The clinical action or decision may comprise recommending the subject for a secondary clinical test to confirm the efficacy of the course of treatment for treating the disease, disorder, or abnormal condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the identifications of ROIs or anatomical structures, a plurality of features extracted from the medical images; and/or clinical health data of the subject between the two or more time points may be indicative of a non-efficacy of the course of treatment for treating the disease, disorder, or abnormal condition of the subject. For example, if the disease, disorder, or abnormal condition was detected in the subject both at an earlier time point and at a later time point, and if the difference is a positive or zero difference (e.g., increased or remained at a constant level from the earlier time point to the later time point), and if an efficacious treatment was indicated at an earlier time point, then the difference may be indicative of a non-efficacy of the course of treatment for treating the disease, disorder, or abnormal condition of the subject. A clinical action or decision may be made based on this indication of the non-efficacy of the course of treatment for treating the disease, disorder, or abnormal condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the disease, disorder, or abnormal condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

### Outputting Reports

After the ROIs or anatomical structures are identified or monitored in the subject, a report may be electronically outputted that is indicative of (e.g., identifies or provides an indication of) a disease, disorder, or abnormal condition of the subject. The subject may not display a disease, disorder, or abnormal condition (e.g., is asymptomatic of the disease, disorder, or abnormal condition, such as a cancer). The report may be presented on a graphical user interface (GUI) of an electronic device of a user. The user may be the subject, a caretaker, a physician, a nurse, or another health care worker.

The report may include one or more clinical indications such as (i) a diagnosis of the disease, disorder, or abnormal condition of the subject, (ii) a prognosis of the disease, disorder, or abnormal condition of the subject, (iii) an increased risk of the disease, disorder, or abnormal condition of the subject, (iv) a decreased risk of the disease, disorder, or abnormal condition of the subject, (v) an efficacy of the course of treatment for treating the disease, disorder, or abnormal condition of the subject, and (vi) a non-efficacy of the course of treatment for treating the disease, disorder, or abnormal condition of the subject. The report may include one or more clinical actions or decisions made based on these one or more clinical indications. Such clinical actions or decisions may be directed to therapeutic interventions, or further clinical assessment or testing of the disease, disorder, or abnormal condition of the subject.

For example, a clinical indication of a diagnosis of the disease, disorder, or abnormal condition of the subject may be accompanied with a clinical action of prescribing a new therapeutic intervention for the subject. As another example, a clinical indication of an increased risk of the disease, disorder, or abnormal condition of the subject may be accompanied with a clinical action of prescribing a new therapeutic intervention or switching therapeutic interventions (e.g., ending a current treatment and prescribing a new treatment) for the subject. As another example, a clinical indication of a decreased risk of the disease, disorder, or abnormal condition of the subject may be accompanied with a clinical action of continuing or ending a current therapeutic intervention for the subject. As another example, a clinical indication of an efficacy of the course of treatment for treating the disease, disorder, or abnormal condition of the subject may be accompanied with a clinical action of continuing or ending a current therapeutic intervention for the subject. As another example, a clinical indication of a non-efficacy of the course of treatment for treating the disease, disorder, or abnormal condition of the subject may be accompanied with a clinical action of ending a current therapeutic intervention and/or switching to (e.g., prescribing) a different new therapeutic intervention for the subject.

### Computer Systems

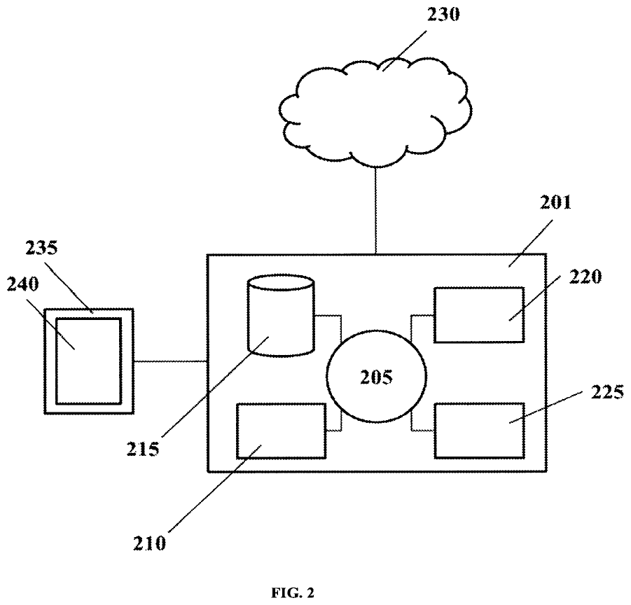

The present disclosure provides computer systems that are programmed to implement methods of the disclosure. FIG. 2 shows a computer system 201 that is programmed or otherwise configured to, for example, train and test a trained algorithm; retrieve a medical image from a remote server via a network connection; identify regions of interest (ROIs) in a medical image; annotate ROIs with label information corresponding to an anatomical structure; generate educational information based at least in part on an annotated medical image; and generate a visualization of an anatomical structure based at least in part on educational information.

The computer system 201 can regulate various aspects of analysis, calculation, and generation of the present disclosure, such as, for example, training and testing a trained algorithm; retrieving a medical image from a remote server via a network connection; identifying regions of interest (ROIs) in a medical image; annotating ROIs with label information corresponding to an anatomical structure; generating educational information based at least in part on an annotated medical image; and generating a visualization of an anatomical structure based at least in part on educational information. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet.

The network 230 in some cases is a telecommunication and/or data network. The network 230 can include one or more computer servers, which can enable distributed computing, such as cloud computing. For example, one or more computer servers may enable cloud computing over the network 230 (“the cloud”) to perform various aspects of analysis, calculation, and generation of the present disclosure, such as, for example, training and testing a trained algorithm; retrieving a medical image from a remote server via a network connection; identifying regions of interest (ROIs) in a medical image; annotating ROIs with label information corresponding to an anatomical structure; generating educational information based at least in part on an annotated medical image; and generating a visualization of an anatomical structure based at least in part on educational information. Such cloud computing may be provided by cloud computing platforms such as, for example, Amazon Web Services (AWS), Microsoft Azure, Google Cloud Platform, and IBM cloud. The network 230, in some cases with the aid of the computer system 201, can implement a peer-to-peer network, which may enable devices coupled to the computer system 201 to behave as a client or a server.

The CPU 205 may comprise one or more computer processors and/or one or more graphics processing units (GPUs). The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, a visual display indicative of training and testing of a trained algorithm; a visual display of a medical image; a visual display of regions of interest (ROIs) in a medical image; a visual display of an annotated medical image; a visual display of educational information of an annotated medical image; and a visualization of an anatomical structure of a subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, train and test a trained algorithm; retrieve a medical image from a remote server via a network connection; identify regions of interest (ROIs) in a medical image; annotate ROIs with label information corresponding to an anatomical structure; generate educational information based at least in part on an annotated medical image; and generate a visualization of an anatomical structure based at least in part on educational information.

### EXAMPLES

### Example 1— Patient Mobile Application For Management and Visualization of Radiological Data

Using systems and methods of the present disclosure, a patient mobile application for management and visualization of radiological data is configured as follows.

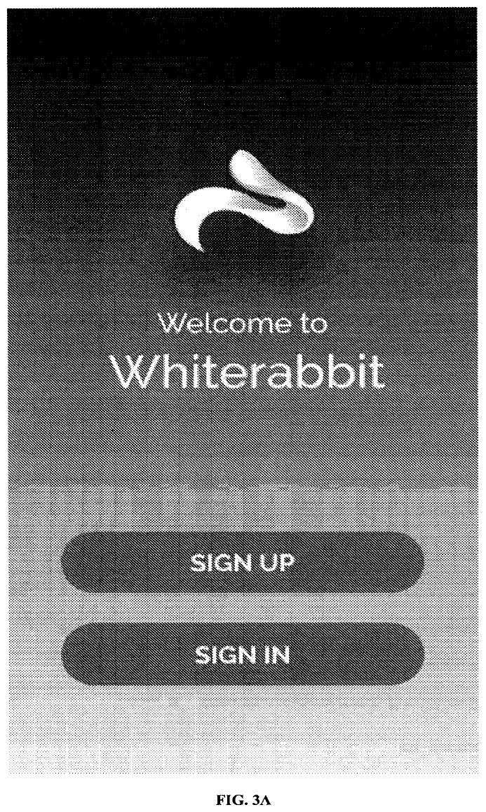

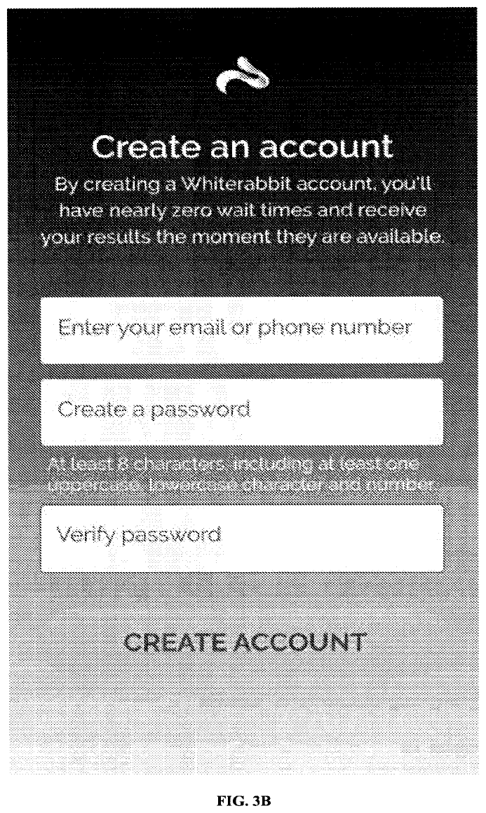

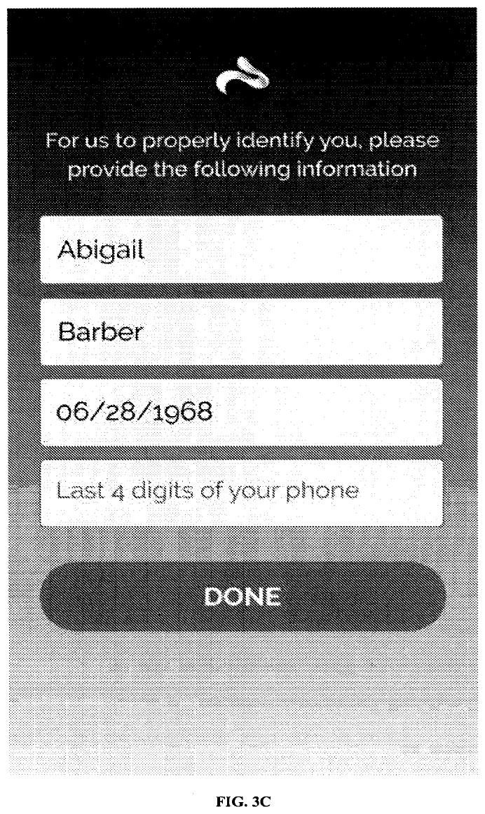

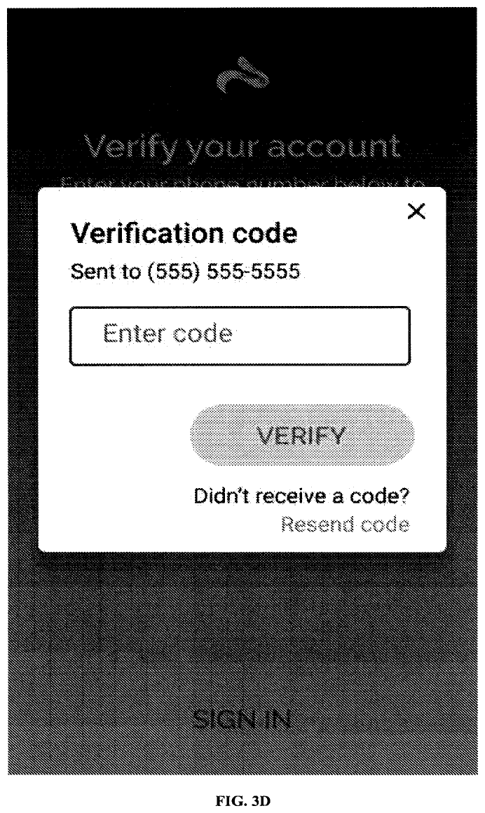

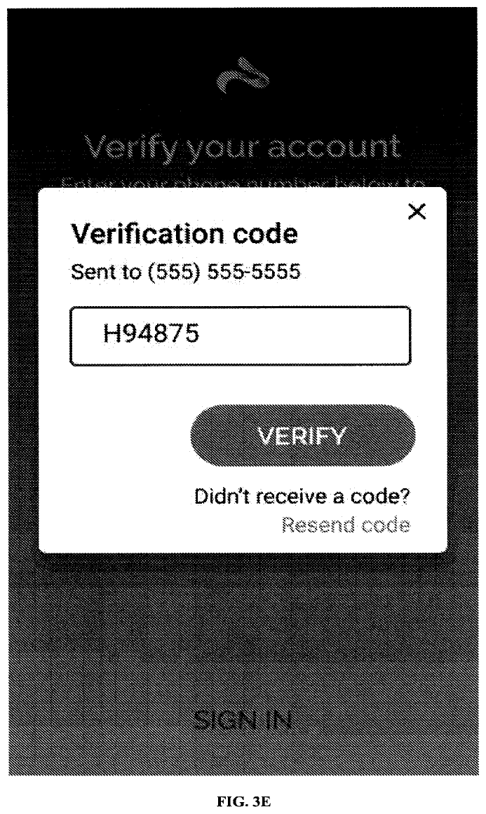

FIG. 3A shows an example screenshot of a mobile application of a radiological data management and visualization system, in accordance with disclosed embodiments. The mobile application is configured to allow a user to participate in the account creation process, which may comprise signing up as a user of the mobile application, or to sign in to the mobile application as an existing registered user of the mobile application.

FIG. 3B shows an example screenshot of a mobile application of a radiological data management and visualization system, in accordance with disclosed embodiments. The mobile application is configured to allow a patient to create a user account of the radiological data management and visualization system, by entering an e-mail address or phone number and creating a password.

FIG. 3C shows an example screenshot of a mobile application of a radiological data management and visualization system, in accordance with disclosed embodiments. The mobile application is configured to allow a user to participate in the patient verification process, which may comprise providing personal information (e.g., first name, last name, date of birth, and last 4 digits of phone number) to identify himself or herself as a patient of an in-network clinic of the radiological data management and visualization system.

FIGS. 3D and 3E show example screenshots of a mobile application of a radiological data management and visualization system, in accordance with disclosed embodiments. The mobile application is configured to authenticate a user by sending a verification code to the user (e.g., through a text message to a phone number of the user) and receiving user input of the verification code.

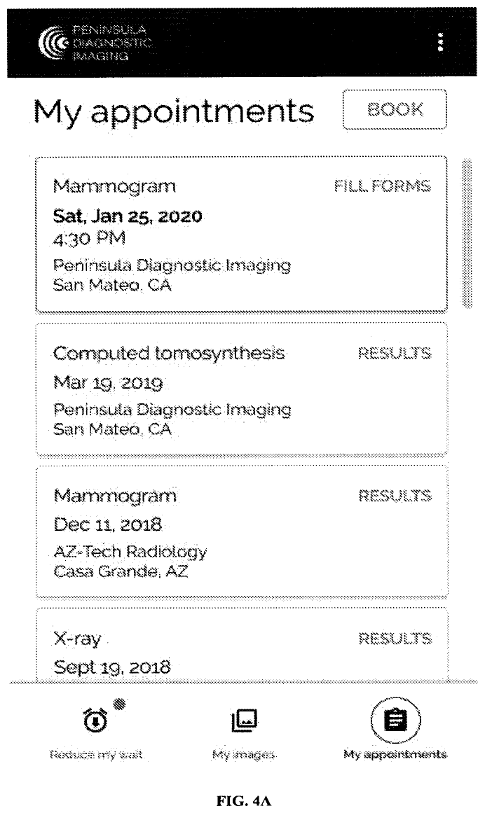

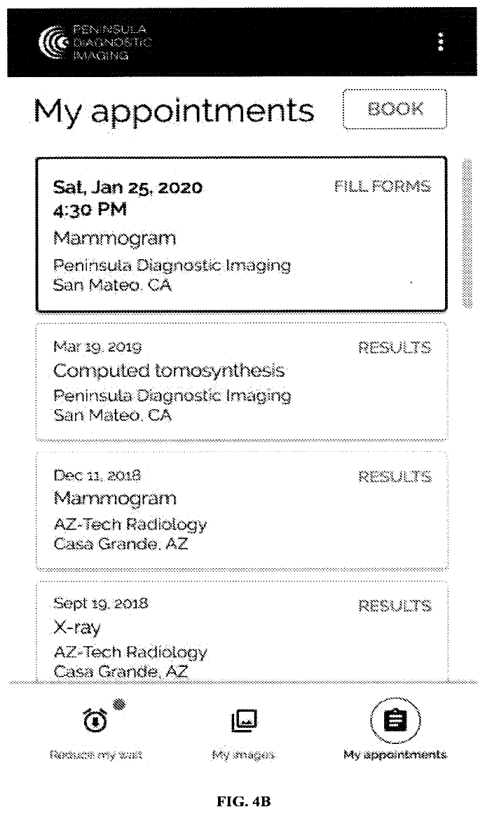

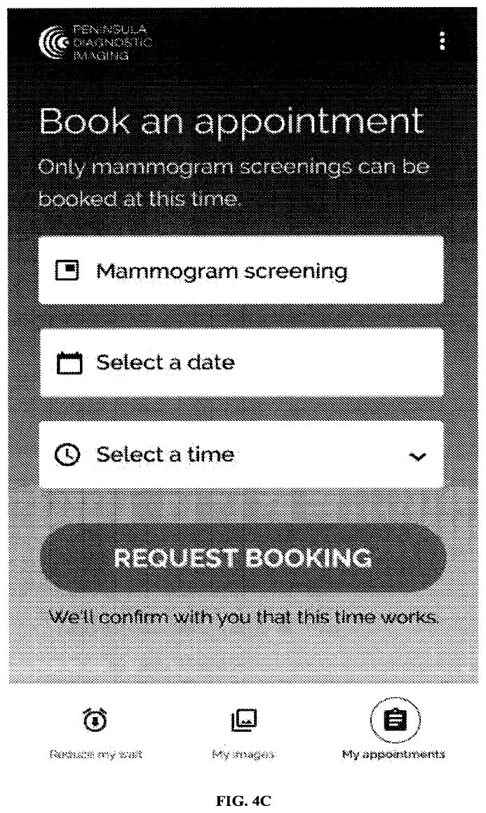

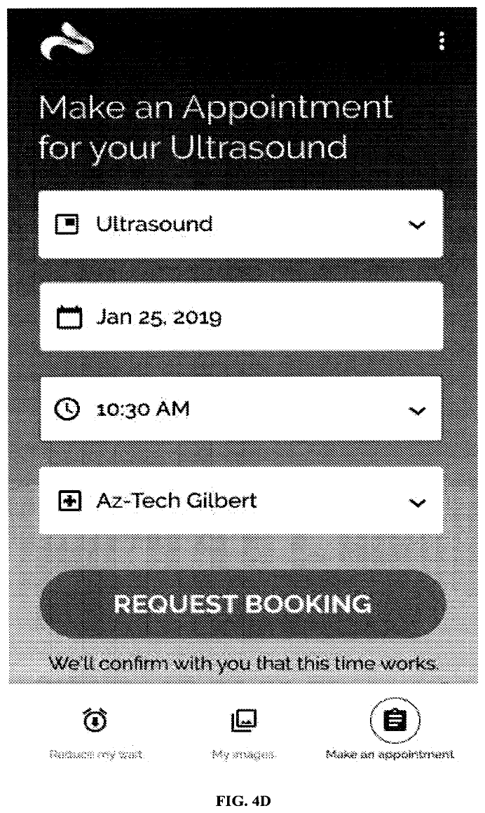

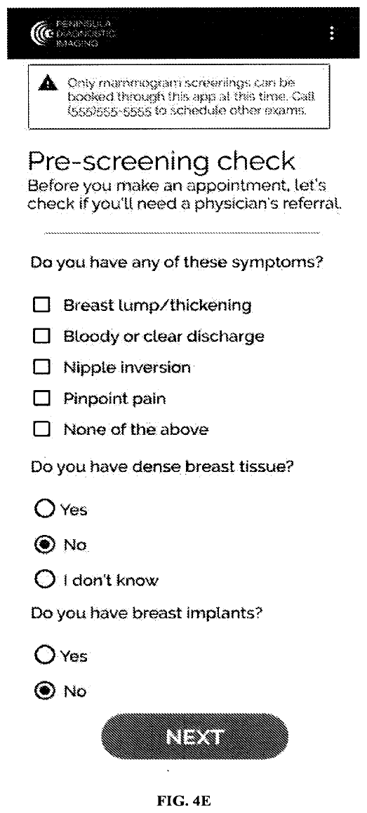

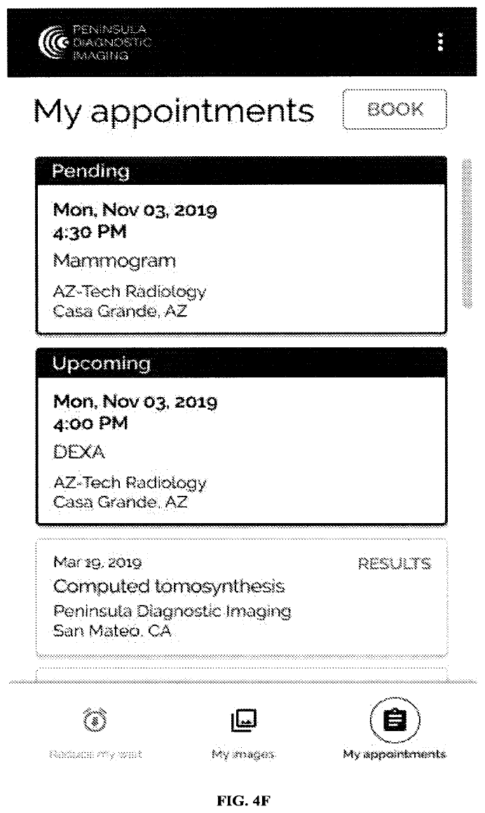

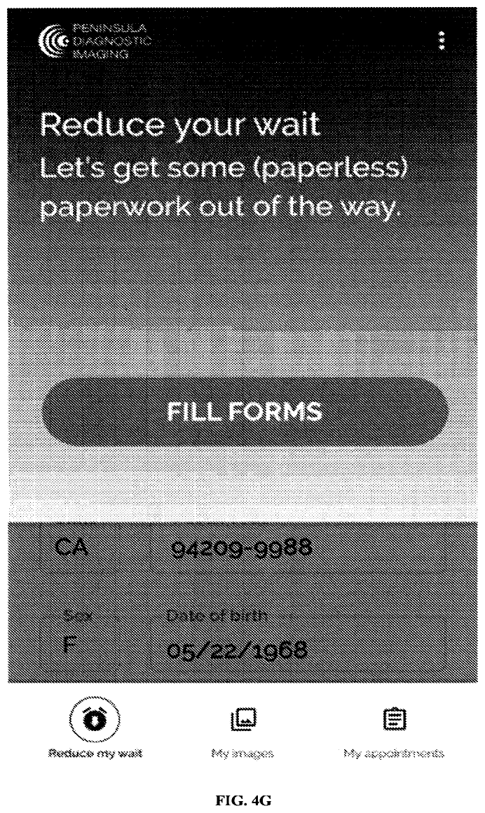

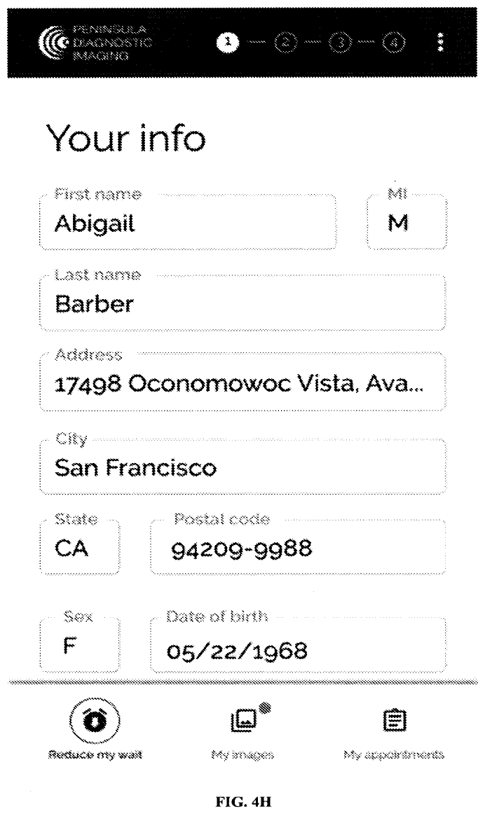

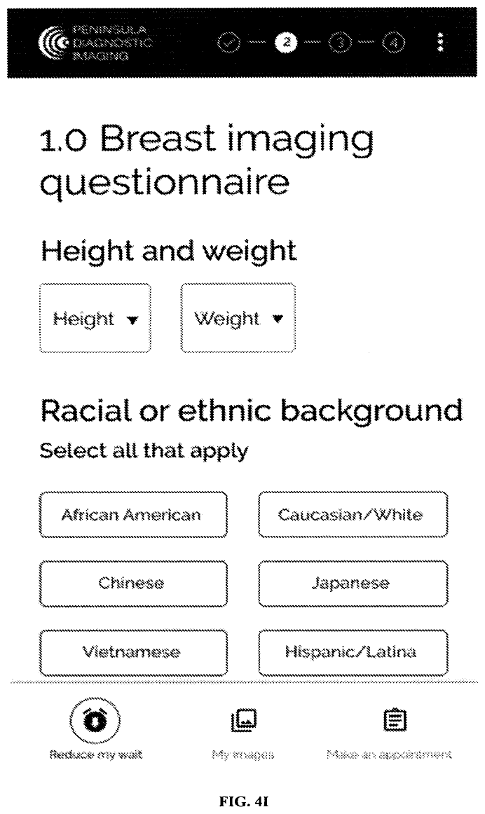

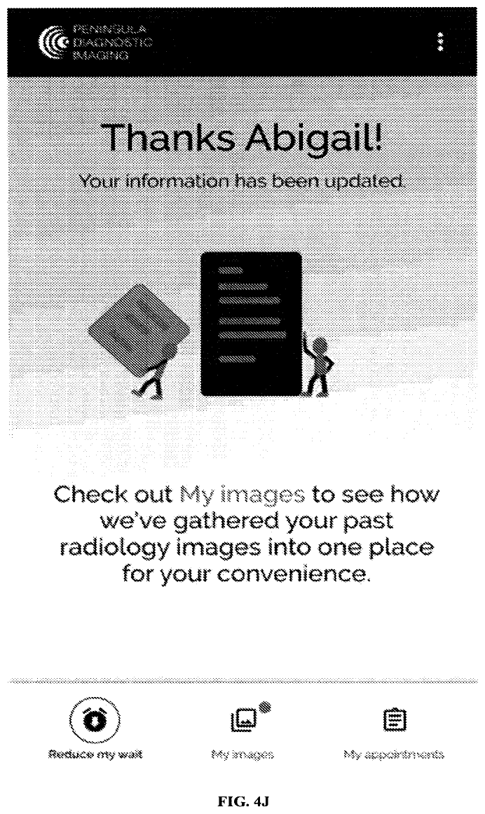

FIG. 4A and 4B show example screenshots of a mobile application of a radiological data management and visualization system, in accordance with disclosed embodiments. The mobile application is configured to allow a user (e.g., a patient) to view a list of his or her appointments. After the user completes the login process, the mobile application may display this “My Appointment” page to the user. All the past and future appointments of the patient with in-network clinics appear on this list. As an example, the list of appointments may include details such as a type of appointment (e.g., mammogram, a computed tomosynthesis, or an X-ray), a scheduled date and time of the appointment, and a clinic location of the appointment. Patients are able to navigate to viewing their results, reports, and images through this page by clicking on that study. For future appointments, the mobile application may allow the user to fill out forms related to the future appointment. For past appointments, the mobile application may allow the user to view the results from the past appointment. In addition, patients are able to request new appointments by clicking “Book.” For reduced waiting, the mobile application is configured to serve the appropriate forms to the patient, including an imaging questionnaire (e.g., breast imaging questionnaire). After the patient has completed the form, the mobile application is configured to confirm the completion of forms and to lead the patient to view the “My Images” page.

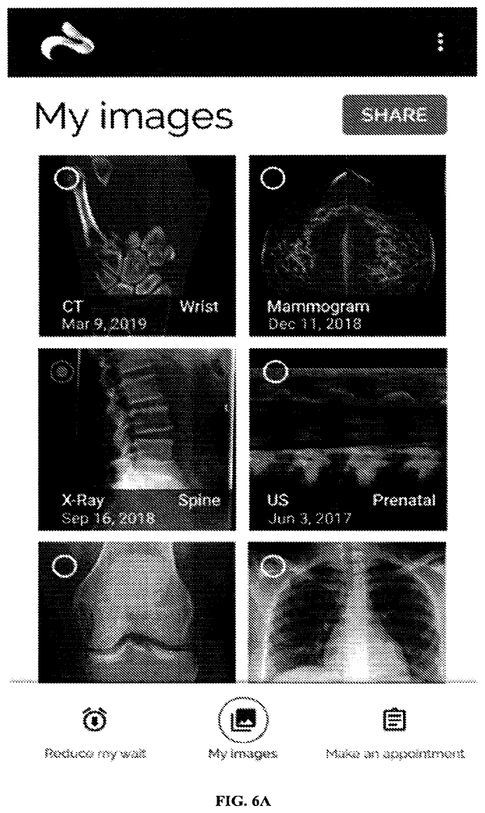

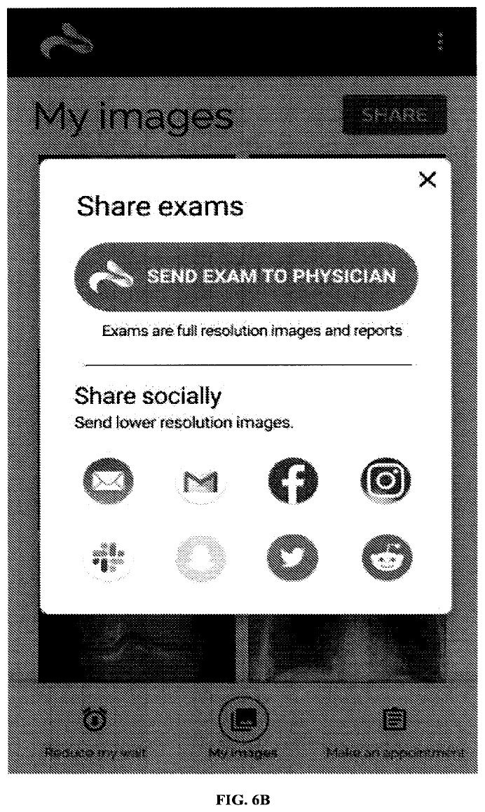

FIGS. 4C and 4D show example screenshots of a mobile application of a radiological data management and visualization system, in accordance with disclosed embodiments. The mobile application is configured to allow a user (e.g., a patient) to book an appointment for radiological assessment (e.g., radiological screening such as mammography). As an example, the mobile application may allow the user to input details of the desired appointment, such as type of appointment (e.g., mammogram screening) and a desired date and time (FIG. 6A). As another example, the mobile application may allow the user to input details of the desired appointment, such as type of appointment (e.g., ultrasound), a desired date and time, and a desired clinic location (FIG. 6B).

FIG. 4E shows an example screenshot of a mobile application of a radiological data management and visualization system, in accordance with disclosed embodiments. The mobile application is configured to allow a patient to participate in a pre-screening check, in which the user is provided a series of questions and is prompted to input response to the series of questions. The questions may include, for example, whether the user has any of a list of symptoms (e.g., breast lump/thickening, bloody or clear discharge, nipple inversion, pinpoint pain, none of the above), whether the user has dense breast tissue, and whether the user has breast implants. Based on the user-provided inputs, the mobile application determines whether the user needs a physician's referral before making an appointment for radiological assessment (e.g., radiological screening such as mammography).

FIG. 4F shows an example of a screenshot of a mobile application of a radiological data management and visualization system, in accordance with disclosed embodiments. The mobile application is configured to allow a user (e.g., a patient) to view a list of his or her appointments. As an example, the list of appointments may include pending appointments and upcoming appointments.

FIGS. 4G-4H show examples of screenshots of a mobile application of a radiological data management and visualization system, in accordance with disclosed embodiments. The mobile application is configured to allow a user (e.g., a patient) to enter his or her personal information (e.g., name, address, sex, and date of birth) into a fillable form. The mobile application may be configured to reduce the wait time of the user by automatically providing the appropriate fillable forms to the user based on an upcoming appointment of the user and/or pre-populating the form's fields with personal information of the user. The mobile application may include a “My Images” button configured to alert the user of new features, such as new fillable forms that are available for an upcoming appointment.

FIG. 4I shows an example of a screenshot of a mobile application of a radiological data management and visualization system, in accordance with disclosed embodiments. The mobile application is configured to present a user (e.g., a patient) with a fillable form (e.g., a questionnaire such as a breast imaging questionnaire) and to allow the user to input information in response to the questionnaire. As an example, the questionnaire may request information of the user, such as height, weight, and racial or ethnic background.

FIG. 4J shows an example of a screenshot of a mobile application of a radiological data management and visualization system, in accordance with disclosed embodiments. The mobile application is configured to present a user (e.g., a patient) with a confirmation that his or her information has been updated, and to link the user to the “My Images” page to view his or her complete record of radiology images.

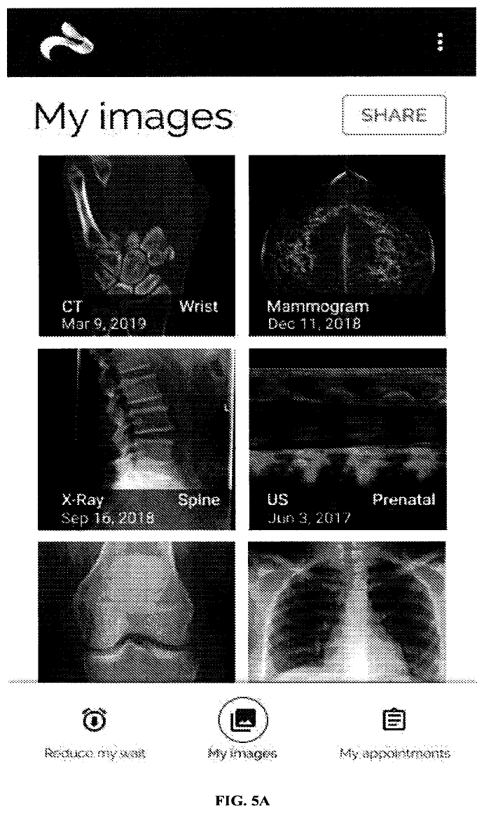

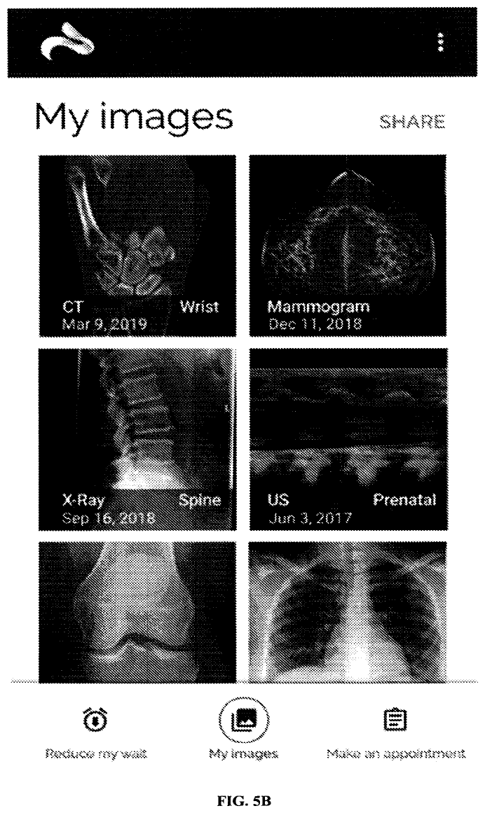

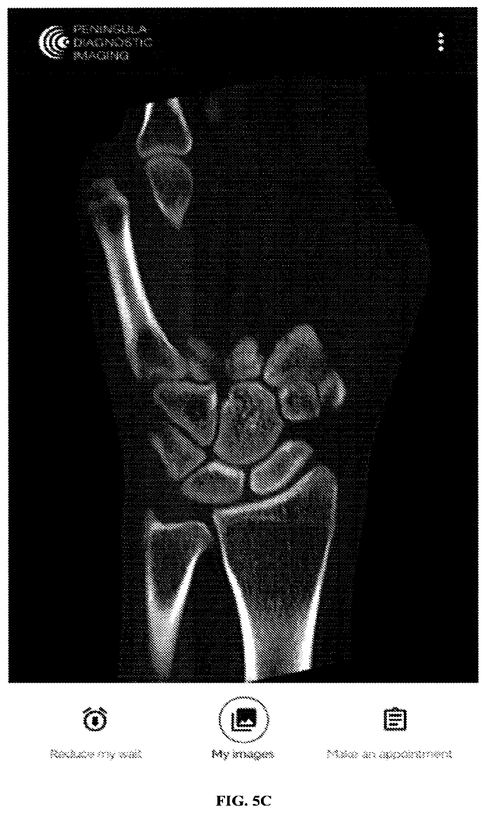

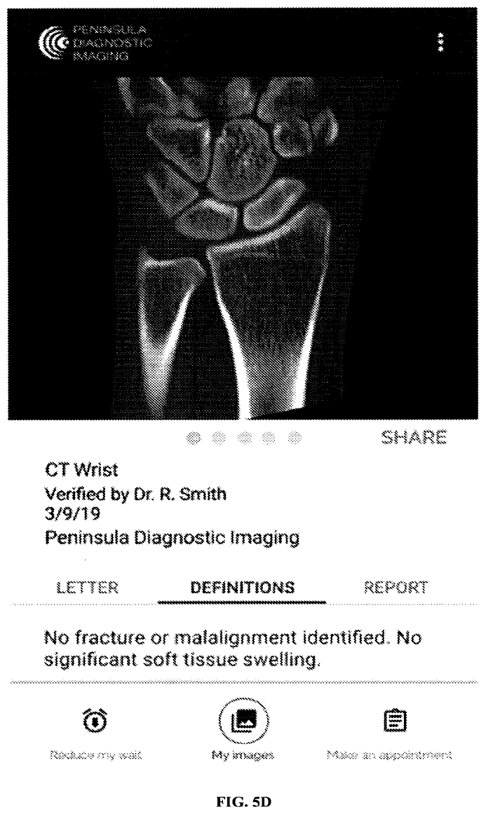

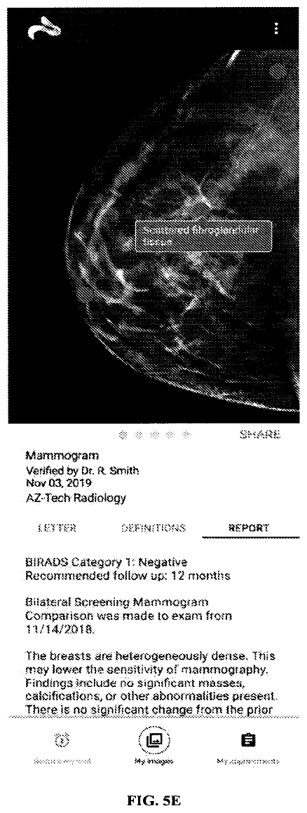

FIG. 5A shows an example of a screenshot of a mobile application of a radiological data management and visualization system, in accordance with disclosed embodiments. The mobile application provides an image viewer configured to allow a user (e.g., a patient) to view sets of his or her medical images (e.g., through a “My Images” page of the mobile application) that have been acquired and stored. As an example, the sets of medical images may be categorized according to an imaging modality (e.g., computed tomography (CT), mammogram, X-Ray, and ultrasound (US)) of the medical images and an acquisition date of the medical images. Each entry of the “My Images” page comprises data associated with an exam visit, and contains multiple images (e.g., medical images acquired), reports, and lay letters. The images are chronologically listed, from most recent to oldest. The thumbnail of each exam shown on the “My Images” page reflects the actual image. The entire plurality of images of a given user is consolidated in a single index, such that the user is able to view his or her entire radiological health record, thereby providing an improved and enhanced user experience and increased convenience and understanding to the user. This may result in further health benefits arising from higher compliance and screening rates for subsequent screening or follow-up care.

FIG. 5B shows an example of a screenshot of a mobile application of a radiological data management and visualization system, in accordance with disclosed embodiments. The mobile application provides an image viewer configured to allow a user (e.g., a patient) to view details of a given medical image upon selection. As an example, for medical images corresponding to 3-dimensional (3-D) exams, the mobile application is configured to present looping GIF files to the user.

FIG. 5C shows an example of a screenshot of a mobile application of a radiological data management and visualization system, in accordance with disclosed embodiments. The mobile application provides an image viewer configured to allow a user (e.g., a patient) to view details of a given medical image upon selection. As an example, to navigate back to the image/exam list, the user taps the “My Images” button.

FIG. 5D shows an example of a screenshot of a mobile application of a radiological data management and visualization system, in accordance with disclosed embodiments. The mobile application provides an image viewer configured to allow a user (e.g., a patient) to view details of a given medical image upon selection. As an example, for each exam, the mobile application uses a carousel to display a plurality of images (e.g., 5 different images). The mobile application also contains tabs for definitions, which include descriptions of various tagged keywords within the report. These definitions are created through a radiologist panel.

FIG. 5E shows an example of a screenshot of a mobile application of a radiological data management and visualization system, in accordance with disclosed embodiments. The mobile application is configured to allow a user (e.g., a patient) to view details of a given medical image that has been acquired and stored, such as annotation options. As an example, the annotations may be present only for a left MLO view of a mammogram. The mobile application may annotate basic anatomy of a view of the medical image, which may comprise identifying one or more anatomical structures of the view of the medical image (e.g., using artificial intelligence-based or machine learning-based image processing algorithms). For example, a view of a medical image of a breast of a subject may be annotated with labels for a fibroglandular tissue, a pectoral muscle, and a nipple. The annotations may have corresponding definitions that are understandable and indicate actionable information for the user.

FIGS. 6A-6B show examples of screenshots of a mobile application of a radiological data management and visualization system, in accordance with disclosed embodiments. The mobile application is configured to allow a user (e.g., a patient) to share his or her exams (e.g., including medical image data and/or reports) to other parties (e.g., physicians or other clinical health providers, family members, or friends), such as by clicking a “Share” button from the “My Images” page. As an example, the user may share the medical image data via e-mail, Gmail, Facebook, Instagram, Twitter, Snapchat, Reddit, or other forms of social media. details of the given medical image may include a letter, definitions, or a medical report (e.g., BIRADS category, recommended follow-up time, comparison to other imaging exams, and a descriptive report of the findings of the imaging exam). The mobile application may be configured to share either full-resolution images or reduced- or low-resolution images with other parties. For example, physicians and clinics may receive full-resolution images, which are packaged specially for medical viewing. As another example, images shared via social media may be converted to reduced- or low-resolution images (e.g., using image compression, image cropping, or image downsampling) before transmission (e.g., to accommodate file size or bandwidth limitations of the social media network).

### Example 2—Patient Mobile Application For Management and Visualization of Radiological Data

Using systems and methods of the present disclosure, a patient mobile application for management and visualization of radiological data is configured as follows.

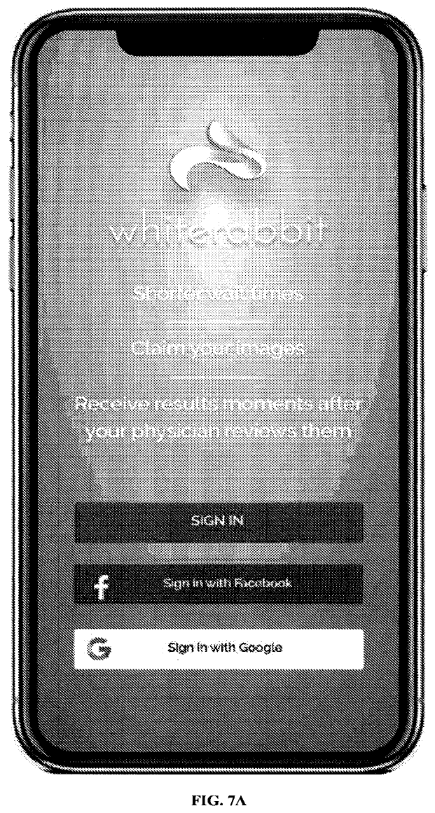

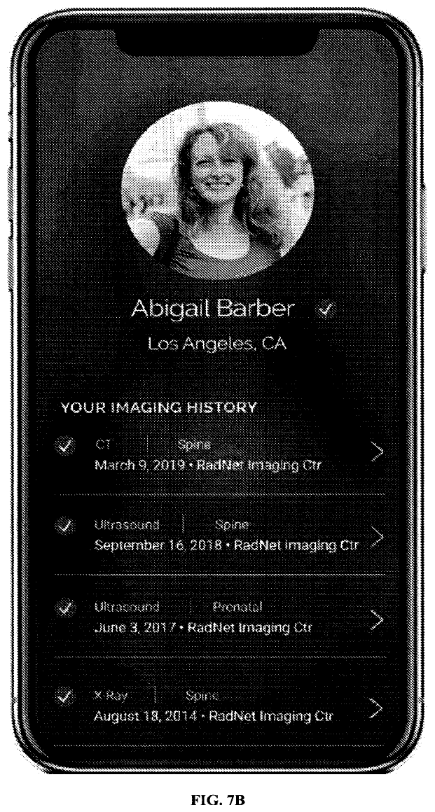

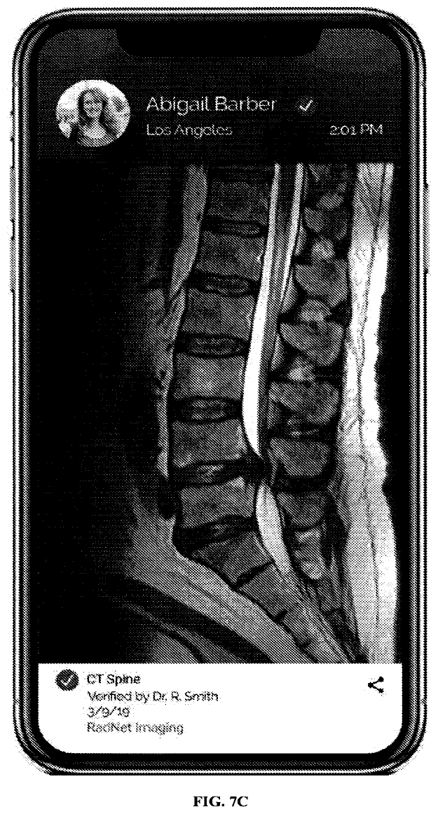

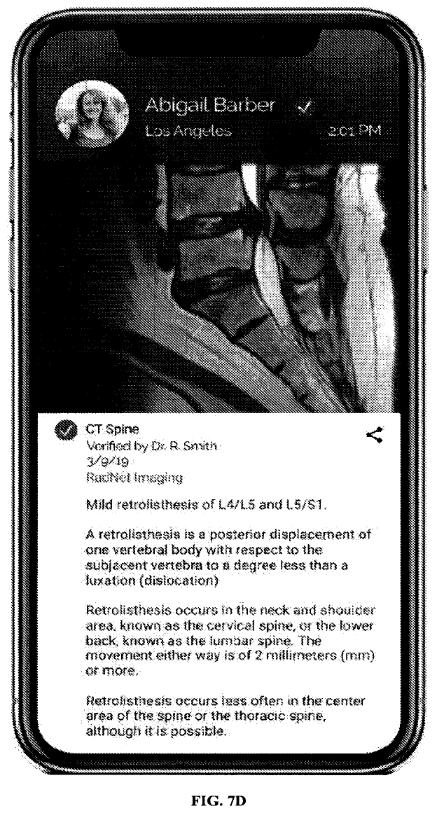

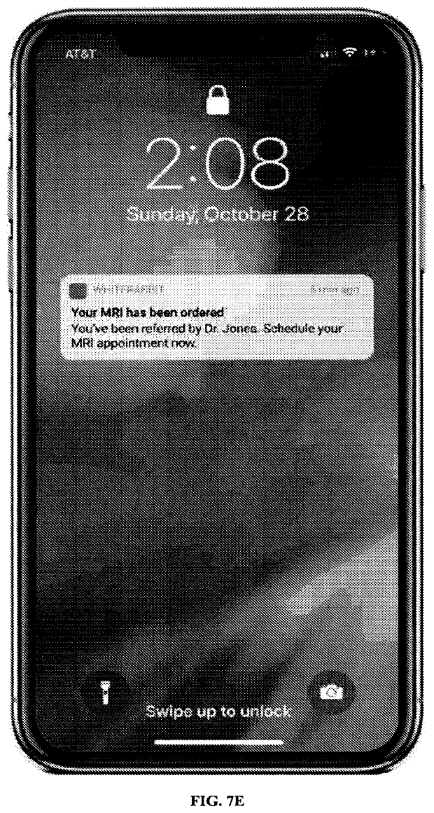

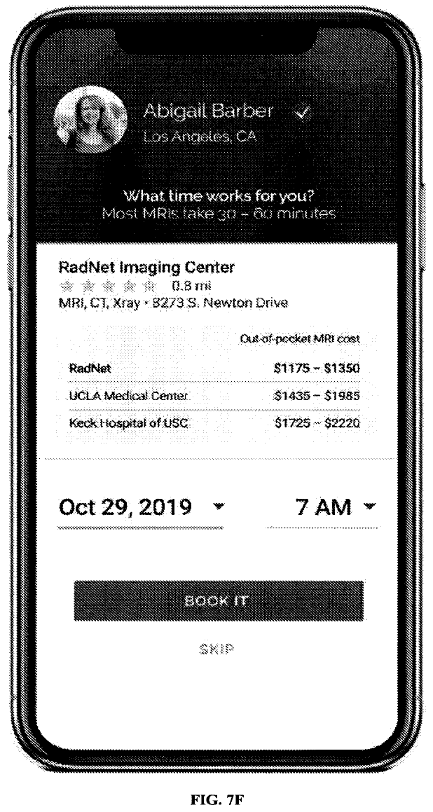

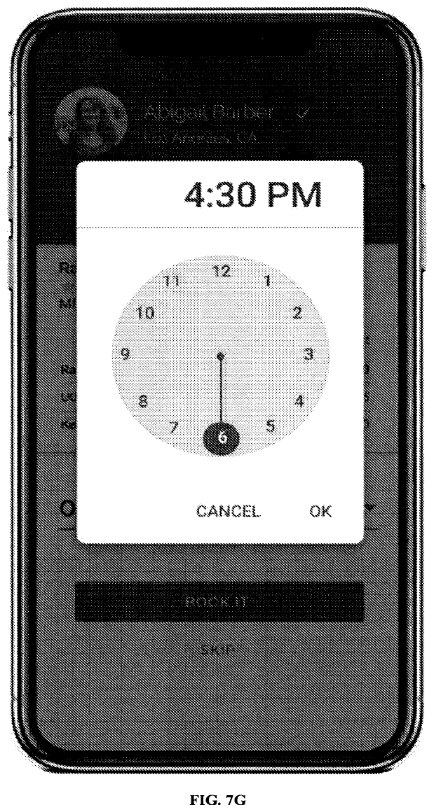

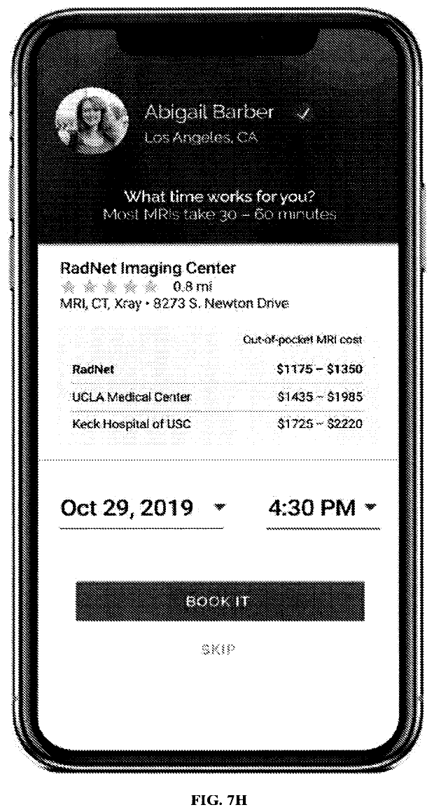

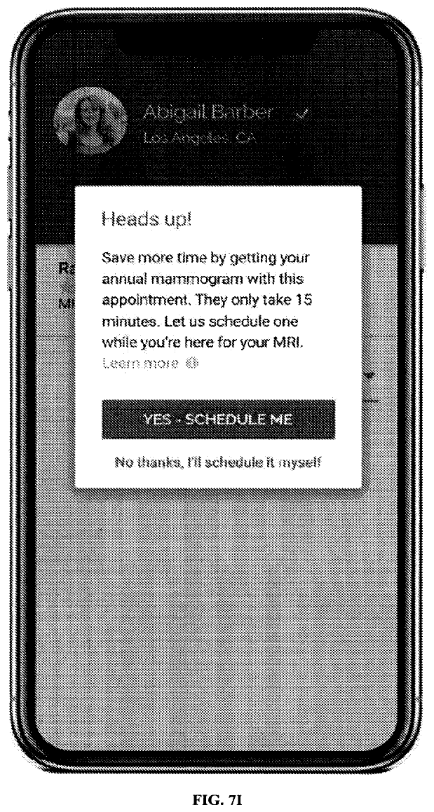

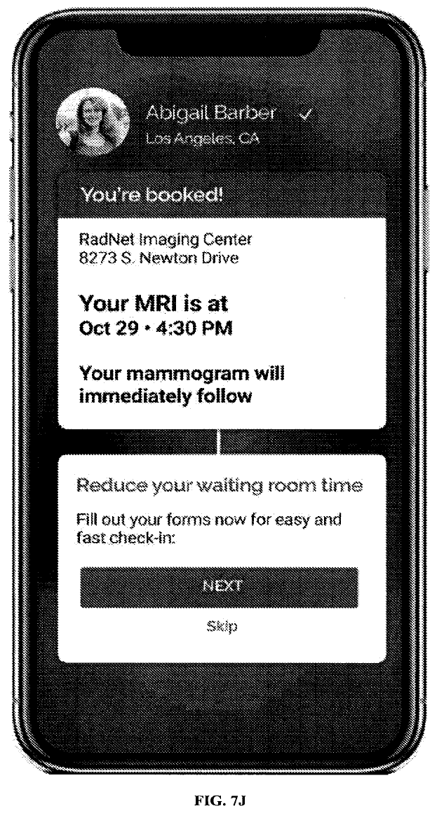

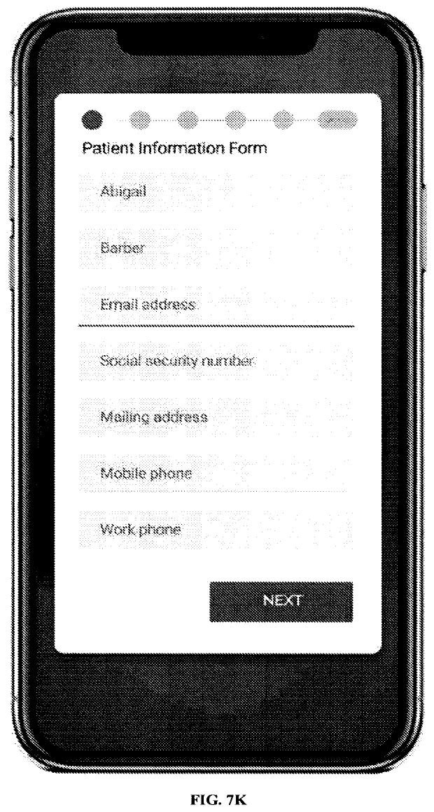

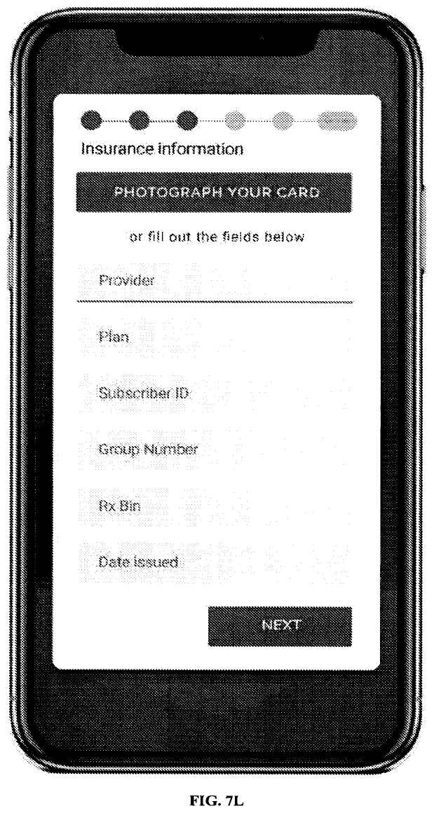

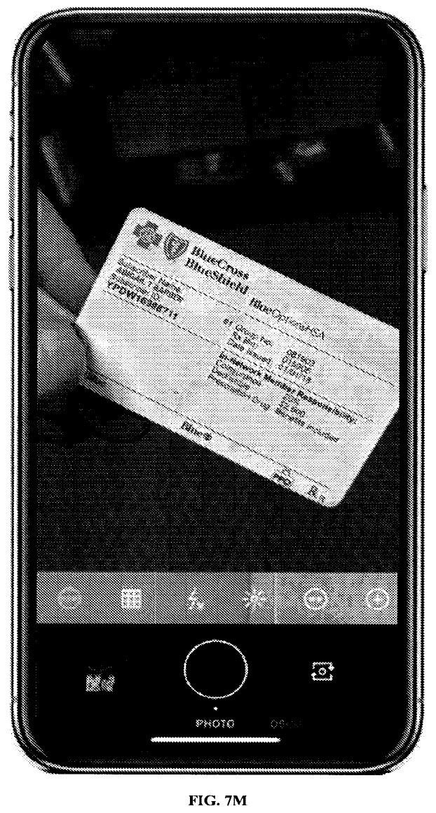

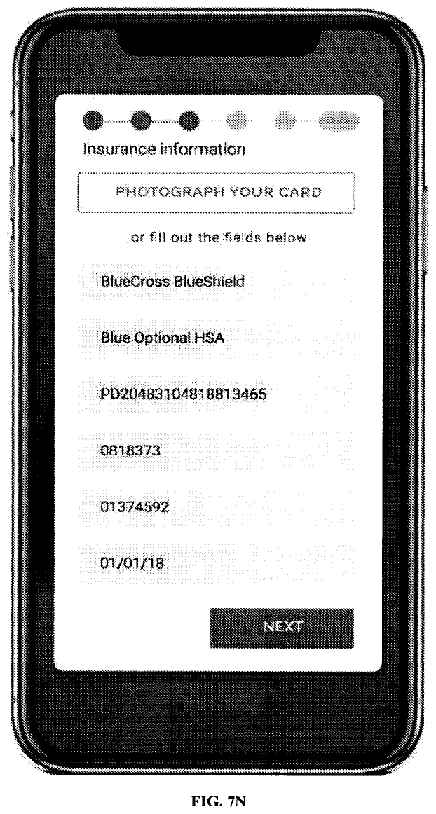

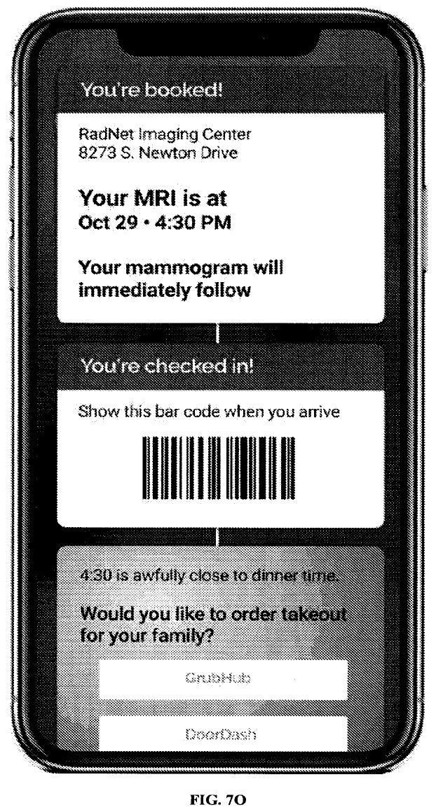

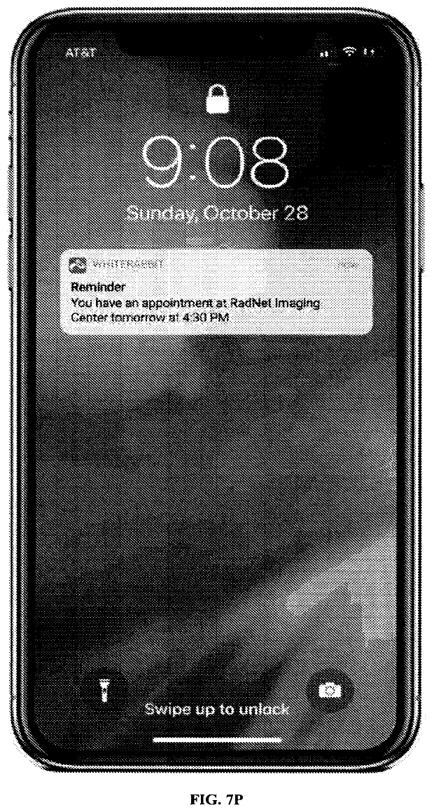

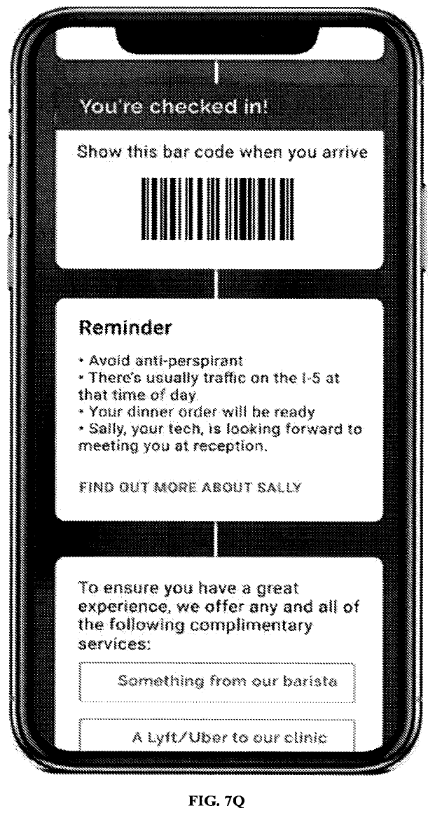

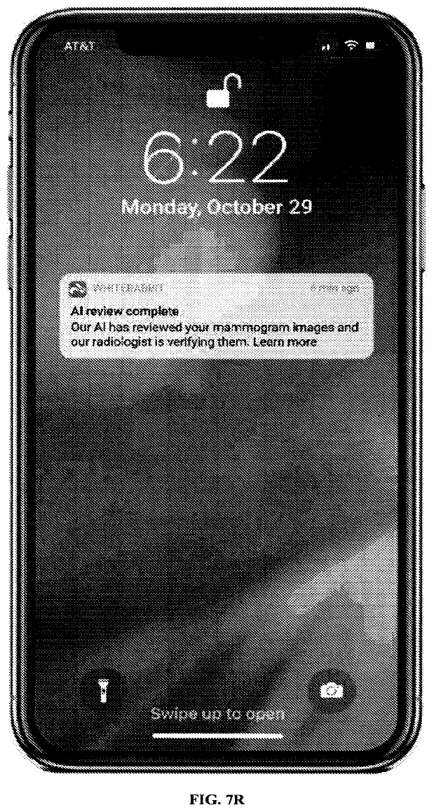

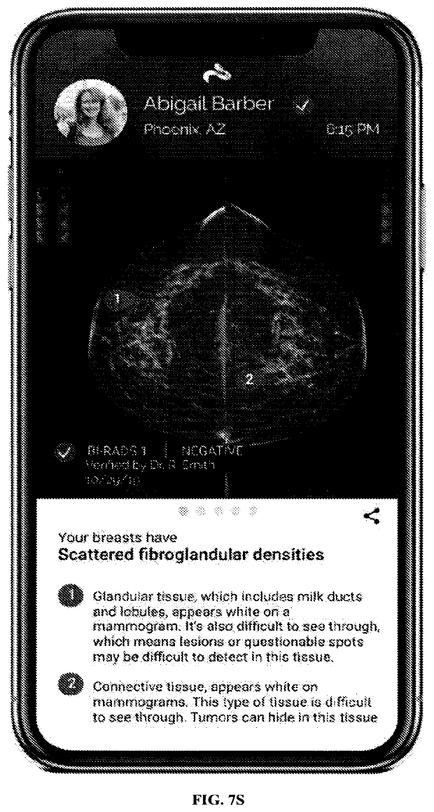

FIGS. 7A-7S show example of screenshots of a mobile application of a radiological data management and visualization system, in accordance with disclosed embodiments. The mobile application is configured to allow a user (e.g., a patient) to book a dual radiological exam (e.g., mammogram and MRI) and facilitate the patient experience throughout the exam process. As an example, the mobile application allows the user to experience shorter wait times, claim his or her images, and receive radiological results moments after his or her physician reviews them (FIG. 7A). As another example, the mobile application allows the user to view a list of his or her entire imaging history, organized by clinical exam visit, including the imaging modality (e.g., CT, ultrasound, X-ray) and location of the body (e.g., spine, prenatal, spine) (FIG. 7B). As another example, the mobile application allows the user to select a clinical exam visit and to view a representative image thereof (FIG. 7C). As another example, the mobile application allows the user to select a clinical exam visit and to view a report summary thereof (FIG. 7D). As another example, the mobile application allows the user to view updates to his or her clinical radiological care, such as when an imaging exam has been ordered or referred by a physician (e.g., primary care physician or radiologist) and when the user is ready to schedule a radiological appointment (FIG. 7E). As another example, the mobile application allows the user to view and select from a plurality of options for a radiological appointment, including details such as date and time, in-network clinic name, and estimated out-of-pocket cost of the imaging exam (FIG. 7F). As another example, the mobile application allows the user to view and select a desired appointment time of the imaging exam (FIG. 7G). As another example, the mobile application allows the user to confirm and book a desired appointment of the imaging exam (FIG. 7H). As another example, the mobile application presents the user with a suggestion to save time by receiving a second radiological exam along with the originally scheduled radiological exam (e.g., a mammogram along with an MRI), and allows the user to select whether or not to schedule the second radiological exam (e.g., a mammogram) (FIG. 7I). As another example, the mobile application presents the user with a confirmation and details of the scheduled appointment of the imaging exam, and with an option to reduce his or her waiting room time by filling out forms for fast and easy check-in (FIG. 7J). As another example, the mobile application presents the user with a patient information form and allows the user to input his or her personal information (e.g., name, Email address, social security number, mailing address, and phone number (FIG. 7K). As another example, the mobile application presents the user with an insurance information form (FIG. 7L) and allows the user to either photograph his or her insurance card (FIG. 7M) or to input his or her insurance information (e.g., provider name, plan, subscriber identification (ID) number, group number, pharmacy (Rx) bin, and date issued) into the form fields (FIG. 7N). As another example, the mobile application presents the user with a confirmation and details of the scheduled appointment of the imaging exam, and a bar code to show when he or she arrives for the scheduled appointment (FIG. 70). As another example, the mobile application presents the user with reminders about his or her scheduled appointment for the imaging exam (FIG. 7P). As another example, the mobile application presents the user with a bar code to show when he or she arrives for the scheduled appointment, and reminders about his or her scheduled appointment for the imaging exam (FIG. 7Q). As another example, the mobile application presents the user with status updates about his or her imaging exam, such as when the exam images have been reviewed (e.g., by a radiologist or artificial intelligence-based method) and/or verified (e.g., by a radiologist) (FIG. 7R). As another example, the mobile application presents the user with imaging exam results, such as a BI-RADS score, an indication of a positive or negative test result, an identification of any test results, such as the presence of suspicious or abnormal characteristics (e.g., scattered fibroglandular densities), and annotated or educational information corresponding to the radiological image (FIG. 7S).

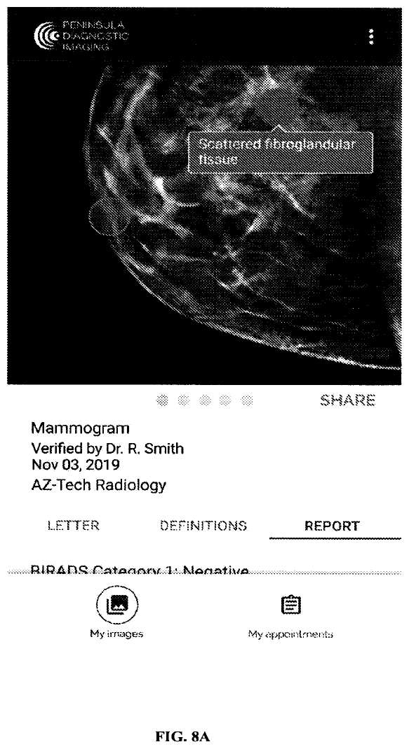

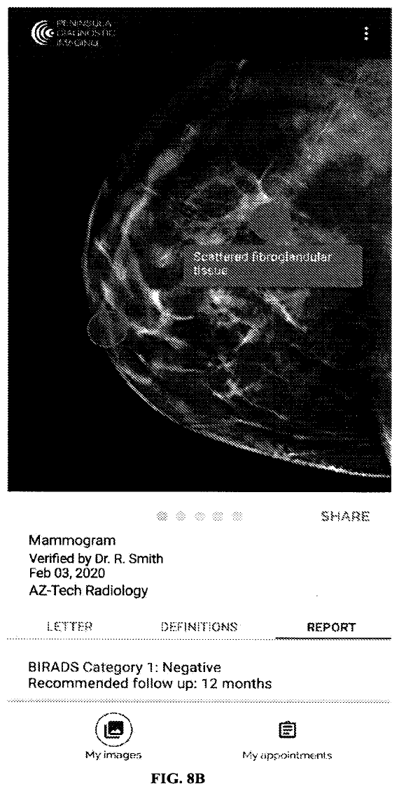

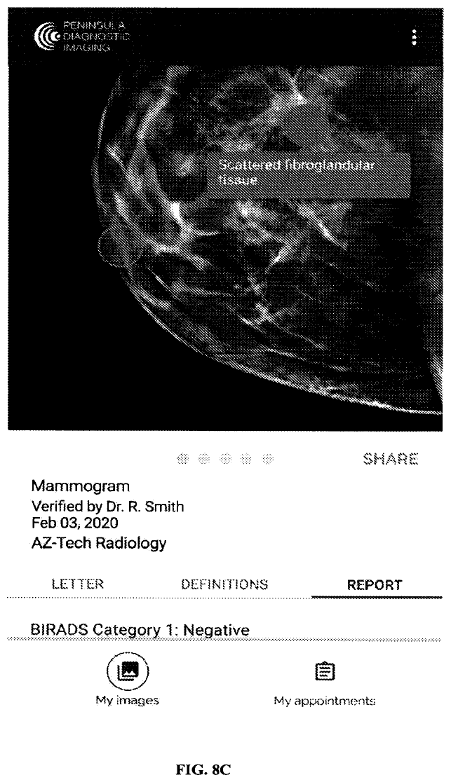

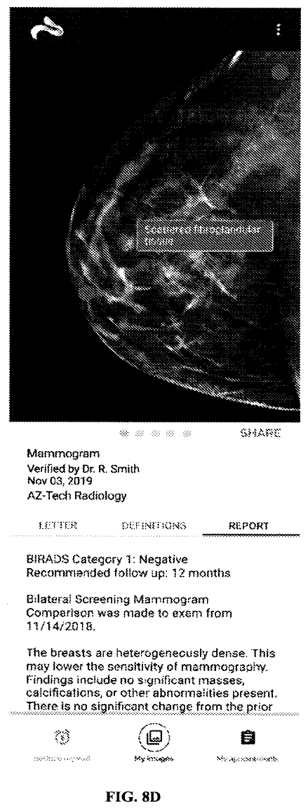

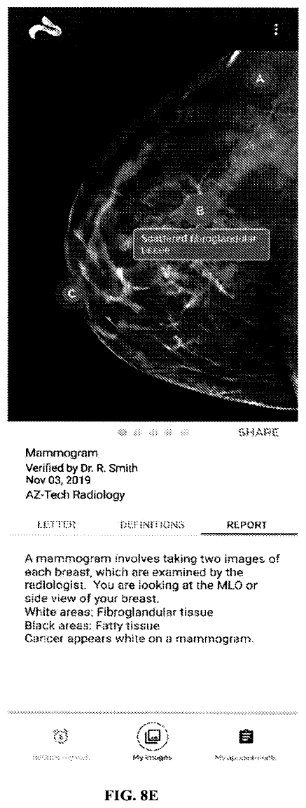

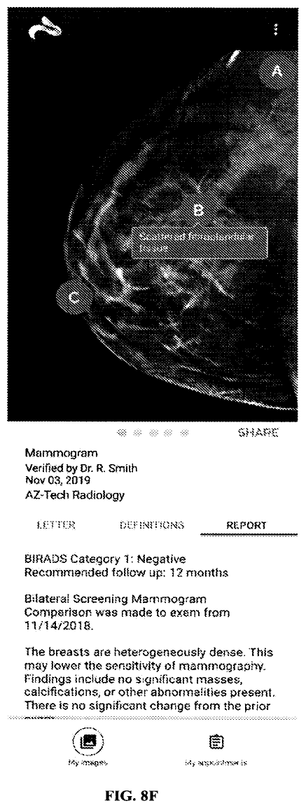

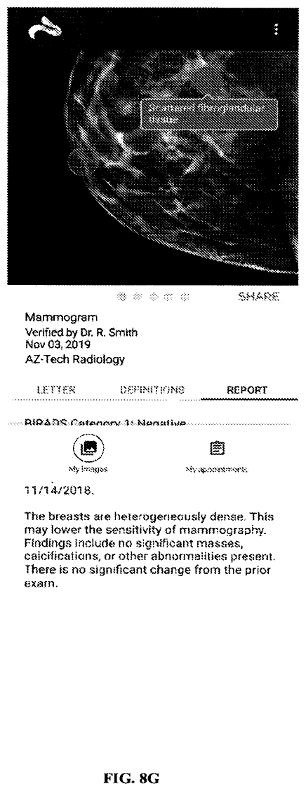

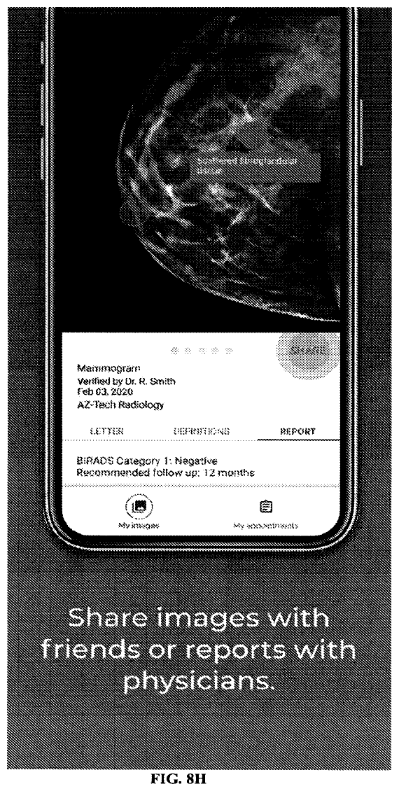

FIGS. 8A-8H show examples of screenshots of a mobile application showing mammogram reports. The mammogram reports may include images of mammogram scans with labeled features, comments from physicians evaluating the scans, and identification information of the evaluating physicians. The labeled features may be abnormalities, e.g., the scattered fibroglandular tissue identified in each of the scans of FIGS. 8A-8H. The features may be labeled (e.g., “A,” “B,” “C,” in FIGS. 8E-F). The labels, or details thereof, may be collapsed or expanded on the interface. For example, a label, or detail thereof, may expand or show upon selection of the labeled feature. Features available for selection may be identified by labels and/or indicators. The reports may indicate whether the user is positive or negative for a condition, e.g., cancer (shown here as BIRADS Category 1). The report may also indicate a suggested follow-up for the patient (e.g., 12 months). The application screens may enable users to view multiple images by swiping or other user interactive actions, and as shown in FIG. 8H, may enable sharing of some or all of the data on the screen with others. The multiple images may be different scan views of scans taken during a particular appointment or may be from scans taken during different appointment. As in FIG. 8D, the reports may contain more detailed comments from physicians or health care professionals. The comment in FIG. 8D explain abnormalities present in the breast tissue. FIG. 8E shows information about what is shown in the image.

### Example 3—Patient Mobile Application For Digital Management of Health Care Appointments

Using systems and methods of the present disclosure, a patient mobile application for digital management of health care appointments for diagnosis, treatment, recovery, and support is configured as follows.

In some embodiments, the patient mobile application for digital management of health care appointments is configured to allow a user to perform one-click booking for routine appointments (e.g., annual check-up or routine screening appointments). In some embodiments, the patient mobile application for digital management of health care appointments is configured to include a platform for patients who are newly diagnosed with a given disease, disorder, or abnormal condition to connect with charities and support groups that are suitable for patients having the given disease, disorder, or abnormal condition. In some embodiments, the patient mobile application for digital management of health care appointments is configured to continually analyze medical images of a user against continually improving trained algorithms (e.g., artificial intelligence-based or machine learning-based models) to generate updated diagnosis results. In some embodiments, the patient mobile application for digital management of health care appointments is configured to include a portal allowing a user to retrieve health care data (e.g., including medical images), store the health care data, and provide access to the health care data (e.g., exchange or share) with other clinical providers, users, friends, family members, or other authorized parties. In some embodiments, the patient mobile application for digital management of health care appointments is configured to include an automated system for tracking the state of progress of a user's exam results. In some embodiments, the patient mobile application for digital management of health care appointments is configured to deliver healthcare reports in a rich multimedia document with medical images and comparisons to population statistics.

### Example 4—Mobile Application For Characterization of Medical Images For Consumer Purposes

Using systems and methods of the present disclosure, a mobile application for characterization of medical images for consumer purposes support is configured as follows.

In some embodiments, the mobile application for characterization of medical images for consumer purposes is configured to use trained algorithms (e.g., artificial intelligence-based or machine learning-based models) to identify anatomy (e.g., anatomical structures) in medical images to educate patients. In some embodiments, the mobile application for characterization of medical images for consumer purposes support is configured to use trained algorithms (e.g., artificial intelligence-based or machine learning-based models) to measure anatomical characteristics to compare to populations of subjects, and to find cohorts of subjects having similar anatomical or clinical characteristics to form social networks thereof. In some embodiments, the mobile application for characterization of medical images for consumer purposes is configured to compute physical dimensions of a subject from medical images of the subject. For example, the mobile application for characterization of medical images for consumer purposes may apply trained algorithms (e.g., artificial intelligence-based or machine learning-based models) to the medical images to determine or estimate physical dimensions of the subject.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for processing at least one medical image of a location of a body of a subject, comprising:
(a) retrieving, from a remote server via a network connection, said at least one medical image of said location of said body of said subject;
(b) identifying one or more regions of interest (ROIs) in said at least one medical image, wherein said one or more ROIs correspond to at least one anatomical structure of said location of said body of said subject;
(c) annotating said one or more ROIs with label information corresponding to said at least one anatomical structure, thereby producing at least one annotated medical image;
(d) generating educational information based at least in part on said at least one annotated medical image; and
(e) generating a visualization of said at least one anatomical structure of said location of said body of said subject, based at least in part on said educational information.

2. The method of claim 1, wherein said at least one medical image is generated by one or more imaging modalities comprising mammography, a computed tomography (CT) scan, a magnetic resonance imaging (MRI) scan, an ultrasound scan, a chest X-ray scan, a positron emission tomography (PET) scan, a PET-CT scan, or any combination thereof.

3. The method of claim 2, wherein said at least one medical image is generated by mammography.

4. The method of claim 3, wherein said location of said body of said subject comprises a breast of said subject.

5. The method of claim 4, wherein said one or more ROIs correspond to a lesion of said breast of said subject.

6. The method of claim 1, wherein said remote server comprises a cloud-based server, and wherein said network connection comprises a cloud-based network.

7. The method of claim 1, wherein (b) comprises retrieving, from said remote server via said network connection, at least one radiological report corresponding to said at least one medical image, and processing said at least one radiological report to identify said one or more ROIs.

8. The method of claim 1, wherein (c) comprises retrieving, from said remote server via said network connection, at least one radiological report corresponding to said at least one medical image, and processing said at least one radiological report to obtain said label information corresponding to said at least one anatomical structure.

9. The method of claim 1, wherein said educational information comprises a location, a definition, a function, a characteristic, or any combination thereof, of said at least one anatomical structure of said location of said body of said subject.

10. The method of claim 9, wherein said location comprises a relative location of said at least one anatomical structure with respect to other anatomical structures of said body of said subject.

11. The method of claim 10, wherein said other anatomical structures of said body of said subject comprise at least a portion or all of an organ system, an organ, a tissue, a cell, or a combination thereof, of said body of said subject.

12. The method of claim 9, wherein said characteristic comprises a density, size, shape, or other measurement of said at least one anatomical structure.

13. The method of claim 1, wherein said educational information comprises diagnostic information, non-diagnostic information, or a combination thereof.

14. The method of claim 13, wherein said educational information comprises non-diagnostic information.

15. The method of claim 1, wherein (e) comprises generating said visualization of said at least one anatomical structure on a mobile device of a user.

16. The method of claim 1, further comprising displaying said visualization of said at least anatomical structure on a display of a user.

17. The method of wherein (b) comprises processing said at least one medical image using a trained algorithm to identify said one or more ROIs.

18. The method of claim 1, wherein (b) comprises processing said at least one medical image using a trained algorithm to identify said at least one anatomical structure.

19. The method of claim 1, wherein (c) comprises processing said one or more ROIs using a trained algorithm to generate said label information and (d) comprises processing said one or more ROIs using a trained algorithm to generate said educational information.

20. The method of claim 17, wherein said trained algorithm comprises a trained machine learning algorithm.

21. The method of claim 20, wherein said trained machine learning algorithm comprises a supervised machine learning algorithm.

22. The method of claim 21, wherein said supervised machine learning algorithm comprises a deep learning algorithm, a support vector machine (SVM), a neural network, or a Random Forest.

23. The method of claim 1, wherein said at least one medical image is obtained via a routine screening of said subject.

24. The method of claim 1, wherein said at least one medical image is obtained as part of a management regimen of a disease, disorder, or abnormal condition of said subject.

25. The method of claim 24, wherein said disease, disorder, or abnormal condition is a cancer.

26. The method of claim 23, wherein said screening is breast cancer screening.

27. The method of claim 1, further comprising storing said at least one annotated medical image in a database.

28. The method of claim 27, further comprising storing said visualization of said at least one anatomical structure in a database.

29. A computer system for processing at least one medical image of a location of a body of a subject, comprising:
a database that is configured to store said at least one medical image of said location of said body of said subject; and
one or more computer processors operatively coupled to said database, wherein said one or more computer processors are individually or collectively programmed to:
(a) retrieve, from a remote server via a network connection, said at least one medical image of said location of said body of said subject;
(b) identify one or more regions of interest (ROIs) in said at least one medical image, wherein said one or more ROIs correspond to at least one anatomical structure of said location of said body of said subject;
(c) annotate said one or more ROIs with label information corresponding to said at least one anatomical structure, thereby producing at least one annotated medical image;
(d) generate educational information based at least in part on said at least one annotated medical image; and
(e) generate a visualization of said at least one anatomical structure of said location of said body of said subject, based at least in part on said educational information.

30-56. (canceled)

57. A non-transitory computer readable medium comprising machine-executable code that, upon execution by one or more computer processors, implements a method for processing at least one medical image of a location of a body of a subject, said method comprising:
(a) retrieving, from a remote server via a network connection, said at least one medical image of said location of said body of said subject;
(b) identifying one or more regions of interest (ROIs) in said at least one medical image, wherein said one or more ROIs correspond to at least one anatomical structure of said location of said body of said subject;
(c) annotating said one or more ROIs with label information corresponding to said at least one anatomical structure, thereby producing at least one annotated medical image;
(d) generating educational information based at least in part on said at least one annotated medical image; and
(e) generating a visualization of said at least one anatomical structure of said location of said body of said subject, based at least in part on said educational information.

58-84. (canceled)
